(19) United States
(12) Patent Application Publication
(10) Pub. No.: US 2022/0133381 A1
(43) Pub. Date: May 5, 2022

(54) DEVICES, SYSTEMS, AND METHODS FOR CRYOABLATION

(71) Applicants: EMORY UNIVERSITY, Atlanta, GA (US); FOCUSED CRYO, INC., Kennesaw, GA (US)

(72) Inventors: John David PROLOGO, Alpharetta, GA (US); Yogi PATEL, Kennesaw, GA (US)

(21) Appl. No.: 17/428,691

(22) PCT Filed: Feb. 10, 2020
(86) PCT No.: PCT/US2020/017453

§ 371 Date: Aug. 5, 2021

Related U.S. Application Data
(60) Provisional application No. 62/802,966, filed on Feb. 8, 2019.
(60) Provisional application No. 62/839,340, filed on Apr. 26, 2019.

(51) Int. Cl. A61B 18/02

(52) U.S. Cl. CPC A61B 18/0218; A61B 2018/00095

(57) ABSTRACT

Device, systems, and methods for cryoablation are described herein. In some implementations, the devices and systems are used to for cryoneurolysis or cryoablation of nerves. An example cryoablation probe includes a tubular member having a proximal end and a distal end. The tubular member has a probe tip arranged at the distal end. The probe also includes one or more energy elements arranged along an axial direction of the tubular member, and one or more sensor elements arranged along the axial direction of the tubular member.

### CROSS-REFERENCE TO RELATED APPLICATIONS

This application claims the benefit of U.S. provisional patent application No. 62/802,966, filed on Feb. 8, 2019, and entitled “SYSTEMS, METHODS, AND OPTIMAL PARAMETERS FOR CRYOABLATION OF NERVES,” and U.S. provisional patent application No. 62/839,340, filed on Apr. 26, 2019, and entitled “TECHNICAL SPECIFICATIONS AND DESCRIPTIONS OF SYSTEMS, METHODS, AND TARGET PARAMETERS FOR FOCUSED CRYOABLATION OF NERVES,” the disclosures of which are expressly incorporated herein by reference in their entireties.

### BACKGROUND

Ablation technologies, such as cryoablation, radiofrequency (RF) ablation, microwave ablation, etc., are common approaches for removing tumors and other undesired tissue structures. The barrier to entry however is that placement of ablation probes near the correct anatomical target is challenging and if placed incorrectly, can result in damage and long-term consequences to the patient. Furthermore, insertion of the probe is conducted blindly without being able to directly observe vessels or other structures in the path to the target tissue. Cryoablation is a common modality used to destroy tumors and other undesired tissue structures. Recently, additional uses and indications for cryoablation are emerging—and when delivered percutaneously using image guidance—represent a blossoming field of minimally invasive needle therapy.

Specifically, the percutaneous application of cold to nerves using closed end needle systems (cryoneurolysis) is a rapidly expanding technique for the management of historically difficult to treat pain syndromes. However, existing cryoablation probes do not allow spatial and temporal control of the ablation zone and lead to damage to non-target tissues, do not provide feedback to the physician on the success of the ablation, and are expensive due to their outdated manufacturing processes. Moreover, conventional systems do not provide the user/operator with the ability to measure local tissue temperatures. Instead, one or more separate temperature probes are inserted to obtain temperature measurements near the surgical site. These temperature probes would be spaced apart from the surgical probe and thus the measurements would only approximate temperature at the targeted nerve. Inserting additional probes may be impractical depending on the anatomy at the surgical site and also increases the risk of injury or infection. Without direct knowledge of the temperature change in the targeted nerve, it is impossible to precisely induce the desired physiological effect (e.g., Wallerian degeneration, Sunderland 2 injury, etc.) and/or make a reasonably educated decision about safety when removing the probe post-procedure.

Essentially, probes configured to destroy target tissue (usually neoplasm) through induction of an osmotic gradient shift, coagulative necrosis, and interspersed apoptosis are being used to ablate nerves without appropriate precision or intent. That is, the mechanism of nerve signal attenuation via decreased temperature is completely separate from the mechanism described above for tumor destruction, and using one for the other is a gross application of existing technology. Specifically, cell death following cryoablation of tumors presently results from freezing induced through a metallic probe cooled with circulated argon. The freeze manifests first in the extracellular space—causing an osmotic gradient to form which leads to cell shrinkage. As the freeze progresses, intracellular ice crystals form and cause damage directly to organelles.

Similar mechanisms result in vascular injury, inducing a coagulative cascade and eventual ischemia mediated cell damage. During the thaw phase of these procedures, water then rushes into previously shrunken cells—causing them to burst. Ablation zone tissues also incur damage through interspersed apoptosis and inflammatory injury. In this setting, precise control of temperature and time are not a priority, as long as the osmotic gradient shift is accomplished and many variable “gross” application protocols have been described.

On the other hand, the mechanism of effect of cryoablation for treatment of conditions related to nerves is drastically different. Precise cold temperatures (e.g., −20° to −100° C.) affect nerves specifically through 1) ice-crystal mediated vasa vasorum damage and endoneural edema, 2) Wallerian degeneration, 3) direct physical injury to axons, and 4) dissolution of microtubules resulting in cessation of axonal transport. The cumulative end point of these routes of neuronal damage is decreased activity resulting from conduction cessation, activation of descending inhibition, blockade of excitatory transmitter systems, and/or generalized sodium channel blockade.

Cryoablation has the potential to treat a myriad of disorders by modulating the nervous system, such as peripheral or autonomic nerves. Cryoablation of nerves has been tested and used however the protocol (e.g., temperature, on time, off time, ramp time) used for targeting nerves is mirrored from the protocol used in cryoablation of tumors. This has clinically led to incomplete ablations and thus complications and side effects for the patient.

Specific technology that provides real time feedback regarding temperature and distance from the probe is therefore needed to allow evidence-based tailoring of protocols for each nerve and condition.

### SUMMARY

Devices, systems, and methods for cryoablation are described herein. These devices, systems, and methods revolutionize clinical cryoablation procedures, at least in part, by including a cryoablation probe that allows for control of the thermal profiles by providing: (1) spatiotemporal control of the thermal gradients and cryoablation zones; and/or (2) real-time (and optionally visual) feedback on the progress and success of the procedure. With the addition of advanced imaging guidance, the potential clinical indications for percutaneous cryoneurolysis can be expanded beyond pain, to include such challenges as premature ejaculation, obesity, or even metabolic conditions. The myriad of nervous system targets in the body allows for a wide spectrum of potential impact.

The devices, systems, and methods described herein can provide feedback regarding the induction of Wallerian degeneration, microtubule dissolution, signal transduction attenuation, and other changes specific to nerve cryoablation through precise, directional temperature manipulation. Such devices, systems, and methods improve patient safety and treatment efficacy. Additionally, such devices, systems, and methods allow an operator/user to know the temperature of the surrounding tissue, which is necessary to safely remove a cryoablation probe following a procedure.

Moreover, the desired effect based on the underlying nature of the nerve and the disease process involved (i.e., autonomic fibers vs. peripheral nerves) require precise, uniform temperature applications for defined amounts of time. For example, the application of cold to nerves for the management of metabolic syndrome or obesity requires precise placement of probes using advanced imaging guidance and specific 2 minute, 1 minute, 2 minute, 1 minute freeze, passive thaw, freeze, passive thaw protocols. Likewise, the management of complex regional pain syndrome can be accomplished through probe placement to the lumbar sympathetic plexi using advanced imaging guidance and specific 2 minute, 1 minute, 2 minute, 1 minute freeze, passive thaw, freeze, passive thaw protocols. Conversely, the management of peripheral neuropathy or pudendal neuralgia (peripheral, mixed nerves) requires precise probe placement and 8 minute, 3 minute, 8 minute, 3 minute protocols 20 to achieve the same effect. The devices, systems, and methods described herein are capable of providing such feedback and control capabilities.

An example cryoablation probe is described herein. The probe includes a tubular member having a proximal end and a distal end. The tubular member has a probe tip arranged at the distal end. The probe also includes one or more energy elements arranged along an axial direction of the tubular member, and one or more sensor elements arranged along the axial direction of the tubular member.

Additionally, each of the one or more energy elements is configured to convert electrical energy to heat. Alternatively or additionally, each of the one or more sensor elements is configured to measure a temperature.

In some implementations, the probe includes a plurality energy elements arranged in a spaced apart relationship along the axial direction of the tubular member. For example, the cryoablation probe can include between about 32 and about 64 energy elements. Optionally, a first group of the energy elements are arranged in a first circumferential region of the tubular member and a second group of the energy elements are arranged in a second circumferential region of the tubular member. Optionally, a first group of the energy elements are arranged in a first axial region of the tubular member and a second group of the energy elements are arranged in a second axial region of the tubular member.

In some implementations, the probe optionally includes a plurality sensor elements arranged in a spaced apart relationship along the axial direction of the tubular member. For example, the cryoablation probe can include between about 32 and about 64 sensor elements.

In some implementations, the one or more energy elements and the one or more sensor elements are arranged within the tubular member. For example, the probe optionally includes a flexible circuit board. The one or more energy elements and the one or more sensor elements are arranged on the flexible circuit board. Optionally, at least a portion of the one or more sensor elements protrude outward from the tubular member. Optionally, the one or more sensor elements are retractable.

In some implementations, the probe tip is a needle. In other implementations, the probe tip has a complex geometry.

In some implementations, the probe includes a fluid channel arranged within the tubular member. The fluid channel is configured to guide a thermally conductive fluid through the tubular member. Additionally, the thermally conductive fluid is liquid or gaseous argon (Ar), liquid or gaseous helium (He), liquid or gaseous hydrogen (H), liquid or gaseous nitrogen (N), or near critical nitrogen (NCN).

In some implementations, the probe includes a handle arranged at the proximal end of the tubular member.

In some implementations, the probe includes an inertial sensor arranged along the axial direction of the tubular member.

In some implementations, the probe includes a light emitter.

In some implementations, the probe includes an inflatable balloon arranged between the proximal and distal ends of the tubular member.

In some implementations, the tubular member is a catheter or a hollow needle.

Another example cryoablation probe is described herein. The probe includes a tubular member having a proximal end and a distal end. The tubular member has a probe tip arranged at the distal end. Additionally the probe includes a fluid channel arranged within the tubular member, wherein the fluid channel is configured to guide a thermally conductive fluid through the tubular member. The probe also includes a temperature sensor element arranged along an axial direction of the tubular member.

Additionally, the temperature sensor element is configured to measure temperature in proximity to the tubular member.

Yet another example cryoablation probe is described herein. The probe includes a tubular member having a proximal end and a distal end. The tubular member has a probe tip arranged at the distal end. The probe also includes an energy element arranged along an axial direction of the tubular member. The energy element is configured to convert electrical energy to heat.

An example cryoablation system is also described herein. The cryoablation system includes a cryoablation probe, a fluid expansion system, and a controller. The cryoablation probe includes a tubular member, a plurality of energy elements, and a plurality of sensor elements. The energy elements and the sensor elements are arranged along an axial direction of the tubular member. The fluid expansion system is arranged at least partially within the tubular member and is configured to circulate a thermally conductive fluid within the tubular member. The controller includes a processor and a memory. The controller is configured to spatially and temporally control a cryoablation zone.

In some implementations, the controller is further configured to spatially and temporally control a plurality of cryoablation zones.

In some implementations, the controller is further configured to individually address each of the energy elements.

In some implementations, the controller is further configured to individually address each of the sensor elements.

In some implementations, the step of spatially and temporally controlling a cryoablation zone includes adjusting a size and/or a shape of the cryoablation zone.

In some implementations, the step of spatially and temporally controlling a cryoablation zone includes selecting an angular region for the cryoablation zone. For example, in some implementations, the angular region is equal to or greater than about a 30° sector in a circumferential direction of the tubular member.

In some implementations, the step of spatially and temporally controlling a cryoablation zone includes steering the cryoablation zone. For example, the cryoablation zone can be rotated in a circumferential direction of the tubular member. Optionally, a direction of rotation can be switched.

In some implementations, the step of spatially and temporally controlling the cryoablation zone includes energizing one or more of the energy elements.

In some implementations, the controller is further configured to receive a measurement detected by at least one of the sensor elements.

In some implementations, the controller is further configured to provide real-time feedback based on the measurement detected by at least one of the sensor elements. Optionally, the real-time feedback is at least one of a visible, audible, or tactile alarm. In some implementations, the system further includes a display device. The controller can be configured to display the real-time feedback on the display device. Optionally, the controller is further configured to energize one or more of the energy elements based on the real-time feedback.

In some implementations, the at least one of the sensor elements is a temperature sensor.

In some implementations, the cryoablation probe further includes an inertial sensor. The controller can be configured to provide information measured by the inertial sensor to a surgical navigation system.

In some implementations, the thermally conductive fluid is liquid or gaseous argon (Ar), liquid or gaseous helium (He), liquid or gaseous hydrogen (H), liquid or gaseous nitrogen (N), or near critical nitrogen (NCN).

An example method is also described herein. The method can include using a cryoablation probe to perform a percutaneous cryoablation procedure on a target tissue, and receiving real-time feedback of local temperature in proximity to the cryoablation probe. The method can also include using the real-time feedback of local temperature in proximity to the cryoablation probe to control the cryoablation probe and destroy the tissue.

Additionally, the local temperature in proximity to the cryoablation probe is measured with a temperature sensor of the cryoablation probe. Optionally, the local temperature is measured within the target tissue at a distance between about 2 millimeters (mm) and about 1 centimeter (cm) from the cryoablation probe.

In some implementations, the target tissue is a nerve. Additionally, the step of using the real-time feedback of local temperature in proximity to the cryoablation probe to control the cryoablation probe and destroy the target tissue includes controlling the cryoablation probe to achieve Wallerian degeneration of the nerve. Wallerian degeneration of the nerve is achieved by controlling the local temperature to achieve a target temperature and/or an amount of time at the target temperature.

In some implementations, the target tissue is a tumor, ganglia, or adipose tissue.

Another example method is described herein. The method includes using a cryoablation probe to perform a percutaneous cryoablation procedure on a target tissue, and receiving real-time feedback of local temperature in proximity to the cryoablation probe. The method also includes using the real-time feedback of local temperature in proximity to the cryoablation probe to control the cryoablation probe and treat a condition.

Additionally, the target tissue is a nerve, tumor, ganglia, or adipose tissue.

Alternatively or additionally, the condition is a metabolic syndrome, type 2 diabetes, hypertension, obesity, sexual dysfunction, chronic pain, phantom limb pain, or a tumor.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a block diagram illustrating an example cryoablation system according to implementations described herein.

FIG. 2 is a diagram illustrating an example cryoablation probe according to implementations described herein.

FIGS. 3A and 3B are diagrams illustrating another example cryoablation probe according to implementations described herein. FIG. 3A is an axial cross section of the probe. FIG. 3B is a radial cross section of the probe.

FIGS. 4A-4D are diagrams illustrating radial cross sections of probes according to implementations described herein. FIG. 4A illustrates a probe achieving a 180° cryozone. FIG. 4B illustrates a probe achieving a 45° cryozone. FIG. 4C illustrates a probe achieving a 270° cryozone. FIG. 4D illustrates a probe achieving a 360° cryozone.

FIG. 5 is a diagram illustrating an example cryoablation probe that is controlled to create a plurality of cryozones according to implementations described herein.

FIG. 6 is a diagram illustrating another example cryoablation probe that is controlled to create a plurality of cryozones according to implementations described herein.

FIG. 7 is illustrates ice block formation around an example cryoablation probe according to implementations described herein.

FIG. 8A is illustrates ice block formation around another example cryoablation probe according to implementations described herein. FIG. 8B is a graph illustrating local temperatures in proximity to the probe of FIG. 8A.

FIG. 9 is an example computing device.

FIG. 10 is a table illustrating time of nerve exposure as it relates to nerve diameter. These are not absolute guaranteed values but are to demonstrate the dependency of exposure duration to nerve diameter.

FIG. 11 is a graph illustrating time of nerve exposure as it relates to nerve diameter. These are not absolute guaranteed values but are to demonstrate the dependency of exposure duration to nerve diameter.

FIG. 12 is a graph illustrating time of nerve exposure as it relates to non-target ablation risk. These are not absolute guaranteed values but are to demonstrate the dependency of exposure duration to non-target ablation risk.

FIG. 13 is an example user interface according to implementations described herein.

FIG. 14A is a CT image showing a conventional cryoablation probe and additional temperature sensing probe inserted into a patient's anatomy during a procedure. FIG. 14B is a graph illustrating tissue temperatures measured by the temperature sensing probe of FIG. 14A.

FIG. 15 is an image illustrating ice block formation around an example cryoablation probe including four flexible circuit boards according to implementations described herein.

FIG. 16 is an axial non-contrast CT image and corresponding cadaveric anatomical model demonstrating the location of critical structures surrounding the pudendal nerve (shaded oval), a target for cryoneurolysis in the setting of pudendal neuralgia or neoplastic pelvic disease.

FIG. 17 illustrates splanchnic nerve cryoablations. Using CT guidance, cryoablation probes (arrows) may be safely navigated around vertebral bodies, the aorta (*), paraspinal arteries, exiting nerve roots, kidneys, the pancreas, the diaphragm, and the celiac artery to target the splanchnic nerves. In this case, though, because the ablation zone was unpredictable and uncontrollable, the diaphragm and paraspinal arteries were at risk.

FIGS. 18A-18D are procedural cryoablation images. Ultrasound and CT guidance were used to place the probe and monitor the ablation, respectively. FIG. 18A is a transverse ultrasound image demonstrates the brachial plexus (black arrows) and associated neuroma (white arrows). FIG. 18B is a longitudinal image in the same region demonstrates the cryoablation probe as it enters the neuroma (arrows). FIG. 18C is an oblique reconstructed intra-procedural CT image demonstrates the hypoattenuating ablation zone (arrows) about the cryoablation probes (stars). FIG. 18D is a corresponding remote pre-procedure T2 weighted MRI image for correlation with FIG. 18C. The hyperintense neuroma is indicated by arrows.

FIG. 19A is a coronal CT image shows unilateral hypertrophic facet arthropathy at C1-C2 (arrowheads). FIG. 19B is an intraprocedural axial CT image shows cryoprobe (*) positioned to include the ipsilateral greater occipital nerve in ablation zone (arrows).

FIG. 20 is an axial CT image from a bilateral pudendal nerve cryoablation procedure demonstrating percutaneous positioning of the probes (arrows) for treatment of pain related to a pelvic mass (arrowhead). See FIG. 16 for anatomic correlation.

FIG. 21A is an axial CT slice, centered on L1, used for body composition assessment. FIG. 21B shows pixel intensities for fat tissue were determined by intensity histogram analysis, and shown as an overlay mask.

FIG. 22A is a dual-axis plot of changes in absolute weight and BMI over time. Error bars represent 95% confidence intervals. FIG. 22B is a dual-axis plot of changes in percentages of total weight loss (TWL), excess weight loss (EWL), and excess BMI loss (EBMIL) over time. Error bars represent 95% confidence intervals.

FIG. 23A are interval plots of changes in Moorehead-Ardelt Questionnaire II scores between pre-procedure and 6 months post-procedure. Error bars represent 95% confidence intervals. FIG. 23B is a comparison of Food Frequency Questionnaire-Derived Daily Dietary Caloric Intake Pre- and Post-Procedure. Error bars represent 95% confidence intervals. *=(p<0.05).

FIG. 24 is an anatomical drawing and corresponding intra-procedural CT image from a splanchnic nerve cryoablation procedure.

FIG. 25 is a CT image demonstrating the position of the cryoablation probe in a male, medial to the pudendal nerve in Alcock's canal.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for cryoneurolysis and/or cryoablation of nerves, it will become evident to those skilled in the art that the implementations are not limited thereto, but are applicable for cryoablation of other tissue types including, but not limited to, tumors, ganglia, and adipose tissue.

Without direct knowledge of the temperature change in a targeted nerve, it is impossible to precisely induce the desired Sunderland 2 injury or make a reasonably educated decisions about safety when removing a cryoablation probe post-procedure. Essentially, probes configured to destroy target tissue (usually neoplasm) through induction of an osmotic gradient shift, coagulative necrosis, and interspersed apoptosis are being used to ablate nerves without appropriate precision or intent. That is, the mechanism of nerve signal attenuation via decreased temperature is completely separate from the mechanism described above for tumor destruction, and using one for the other is a gross application of existing technology. Instead, probes that provide feedback regarding the induction of Wallerian degeneration, microtubule dissolution, signal transduction attenuation, and other changes specific to nerve cryoablation through precise, directional temperature manipulation are needed for patient safety and improved efficacy. Finally, in order to safely remove a cryoablation probe following a procedure, it is necessary to know the temperature of the surrounding tissue. Otherwise adjacent and target tissues can be significantly damaged upon removal of the probe.

As described above, cryoablation of nerves has been tested and used however the protocol (e.g., temperature, on time, off time, ramp time) used for targeting nerves is mirrored from the protocol used in cryoablation of tumors. This has clinically led to incomplete ablations and thus complications and side effects for the patient. Specifically, cell death following cryoablation of tumors presently results from freezing induced through a metallic probe cooled with circulated argon. The freeze manifests first in the extracellular space—causing an osmotic gradient to form which leads to cell shrinkage. As the freeze progresses, intracellular ice crystals form and cause damage directly to organelles.

Similar mechanisms result in vascular injury, inducing a coagulative cascade and eventual ischemia mediated cell damage. During the thaw phase of these procedures, water then rushes into previously shrunken cells—causing them to burst. Ablation zone tissues, which are not the intended target, also incur damage through interspersed apoptosis and inflammatory injury. In this setting, precise control of temperature and time are not a priority, as long as the osmotic gradient shift is accomplished—and many variable “gross” application protocols have been described.

On the other hand, the mechanism of effect of cryoablation for treatment of conditions related to nerves is drastically different. Precise cold temperatures (−20 to −100 C) affect nerves specifically through: (1) ice-crystal mediated vasa vasorum damage and endoneural edema; (2) Wallerian degeneration; (3) direct physical injury to axons, and (4) dissolution of microtubules resulting in cessation of axonal transport.

The cumulative end point of these routes of neuronal damage is decreased activity resulting from conduction cessation, activation of descending inhibition, blockade of excitatory transmitter systems, and/or generalized sodium channel blockade. The desired effect based on the underlying nature of the nerve and the disease process involved—i.e., autonomic fibers vs. peripheral nerves—require precise, uniform temperature applications for defined amounts of time.

The application of cold to nerves for the management of metabolic syndrome or obesity requires precise placement of probes using advanced imaging guidance and specific freeze/thaw protocols. Likewise, the management of complex regional pain syndrome can be accomplished through probe placement to the lumbar sympathetic plexi using advanced imaging guidance and specific freeze/thaw protocols. Conversely, the management of peripheral neuropathy or pudendal neuralgia (peripheral, mixed nerves) requires precise probe placement and respective freeze/thaw protocols. Specific technology that provides real time feedback regarding temperature and distance from the probe facilitates evidence-based tailoring of these protocols for each nerve and condition, to include seizures, obesity, diabetes, hypertension, metabolic syndrome, sexual disorders, and central and peripheral pain syndromes. In all cases, the effect of treating these various disorders by cryoablating nerves takes place by inducing Wallerian degeneration, which achieves a resetting of the neural circuit over the course of weeks to months—ending in treatment of the disorder(s).

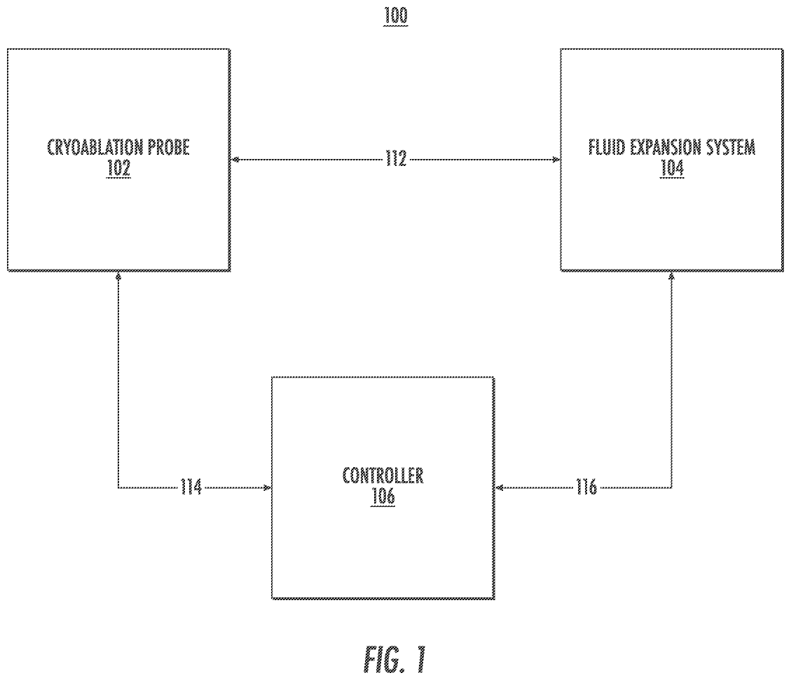

Referring now to FIG. 1, an example cryoablation system 100 is shown. The cryoablation system 100 includes a cryoablation probe 102, a fluid expansion system 104, and a controller 106. Cryoablation uses extreme cold (e.g., −20° C. to −100° C.) to destroy target tissue. As described herein, in some implementations, the target tissue is a nerve. Cryoablation is performed by circulating thermally conductive fluid through the cryoablation probe 102, which is positioned near the target tissue. Example thermally conductive fluids are liquid or gaseous argon (Ar), helium (He), hydrogen (H), liquid or gaseous nitrogen (N), or near critical nitrogen (NCN). It should be understood that the fluids described above are provided only as examples. This disclosure contemplates using other thermally conductive fluids with the system 100 described herein. The cryoablation probe 102 and the fluid expansion system 104 are in fluid connection, which is shown by reference number 112.

The fluid expansion system 104 can include a refrigerated fluid reservoir, a pump, and inlet and return channels. The fluid expansion system 104 is configured to circulate the thermally conductive fluid within the cryoablation probe 102. The thermally conductive fluid is delivered, for example via an inlet channel, to the cryoablation probe 102. As the thermally conductive fluid traverses through the inlet channel, the fluid expansion system 104 is designed such that the thermally conductive fluid expands (e.g., using an expansion chamber), which causes temperature to decrease. This is how the extremely cold temperatures are achieved. The thermally conductive fluid is then returned to the fluid reservoir via a return channel. A pump can be used to move the thermally conductive fluid through the fluid expansion system 104. It should be understood that at least a portion of the fluid expansion system 104 is arranged within the cryoablation probe 102. For example, as described above, the thermally conductive fluid is delivered to the cryoablation probe 102, where such fluid undergoes expansion before returning to the fluid reservoir. Accordingly, in some implementations, at least portions of the inlet and return channels are arranged within the cryoablation probe 102. This disclosure contemplates that other fluid expansion system 104 components can be integrated with the cryoablation probe 102.

The system 100 also includes the controller 106, which includes a processor and a memory. In some implementations, the controller 106 can be a computing device as shown in FIG. 9. The controller 106 can be operably connected to the cryoablation probe 102. For example, the controller 106 and the cryoablation probe 102 can be connected by a communication link 114. As described herein, the controller 106 is configured to spatially and temporally control a cryoablation zone. As used herein, a cryoablation zone (sometimes referred to as “cryozone”) is the region in proximity to the probe 102 where ice forms in a subject's body as a result of the low temperatures of the probe 102. In some implementations, the controller 106 is also operably connected to the fluid expansion system 104. For example, the controller 106 and the fluid expansion system 104 can be connected by a communication link 116. This disclosure contemplates the communication links are any suitable communication link. For example, a communication link may be implemented by any medium that facilitates data exchange including, but not limited to, wired, wireless and optical links. Example communication links include, but are not limited to, a local area network (LAN), a wireless local area network (WLAN), a wide area network (WAN), a metropolitan area network (MAN), Ethernet, the Internet, or any other wired or wireless link such as WiFi, WiMax, 3G, 4G, or 5G.

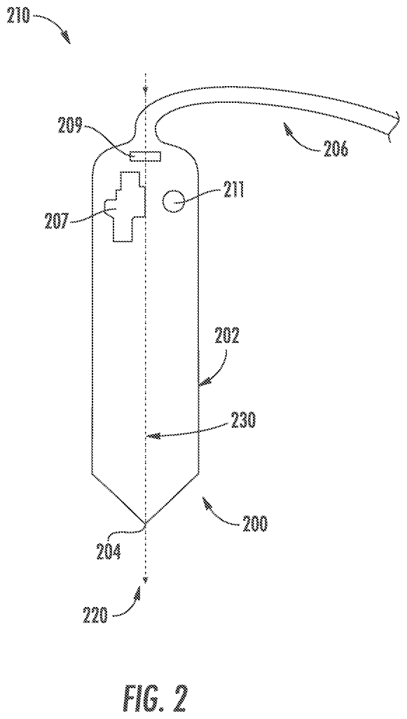

Referring now to FIG. 2, an example cryoablation probe 200 is shown. The probe 200 includes a tubular member 202 having a proximal end 210 and a distal end 220. The tubular member 202 has a probe tip 204 arranged at the distal end 220. Optionally, the tubular member 202 is a catheter or a hollow needle. In some implementations, the probe tip 204 is a needle. In other implementations, the probe tip 204 has a complex geometry to accommodate the target structure. A handle is arranged at the proximal end 210 of the tubular member 202. As described herein, the probe 200 can also include one or more energy elements (not shown in FIG. 2) and one or more sensor elements (not shown in FIG. 2). The energy element(s) and the sensor element(s) are arranged along an axial direction 230 of the tubular member 202. As shown in FIG. 2, a tube 206 is used to couple the probe 200 to external systems, for example, a fluid expansion system and/or a controller as described above with regard to FIG. 1. The tube 206, which can be insulated, may include wires and/or fluid channels therein. Optionally, the probe 200 can include a computer-readable identifier 207. For example, the identifier 207 can be a barcode (one-dimension or two-dimensional), a radiofrequency identification (RFID) tag, or other computer-readable marker. In some implementations, the probe 200 can include a plurality of identifiers. The identifier 207 is capable of being scanned (e.g., optical, magnetic, electromagnetic, etc.) and then read/decoded with a computer. The identifier 207 can be provided on an external surface of the probe 200. It should be understood that the location of the identifier 207 on the probe 200 in FIG. 2 is provided only as an example. This disclosure contemplates that the identifier can be used for identification and/or tracking of the probe 200. Alternatively or additionally, the probe 200 can optionally include embedded electronics 209. This disclosure contemplates that the embedded electronics 209 can be used for warranty, access, use control, etc. It should be understood that the location of the embedded electronics 209 on the probe 200 in FIG. 2 is provided only as an example. Alternatively or additionally, the probe 200 can optionally include a light emitter 211. For example, the light emitter 211 can be a light-emitting diode (LED). In some implementations, the probe 200 can include a plurality of light emitters. This disclosure contemplates that the light emitter 211 can be provide feedback control to a user/operator of the probe 200. The light emitter(s) can be used to show probe status to the user/operator. For example, the light emitter(s) may light up in sequence to show “progress”. Alternatively or additionally, the light emitter(s) may flash and then stay lit to show the user/operator status of the therapy at a distance. This disclosure contemplates that the light emitter(s) can be the same and/or different colors. It should be understood that the location of the light emitter 211 on the probe 200 in FIG. 2 is provided only as an example. Alternatively or additionally, the probe 200 may be compatible with surgical imaging modalities including, but not limited to, CT, magnetic resonance imaging (MRI), or ultrasound. This can be achieved by constructing the probe 200 with suitable materials and/or providing imaging compatible coatings on the probe 200.

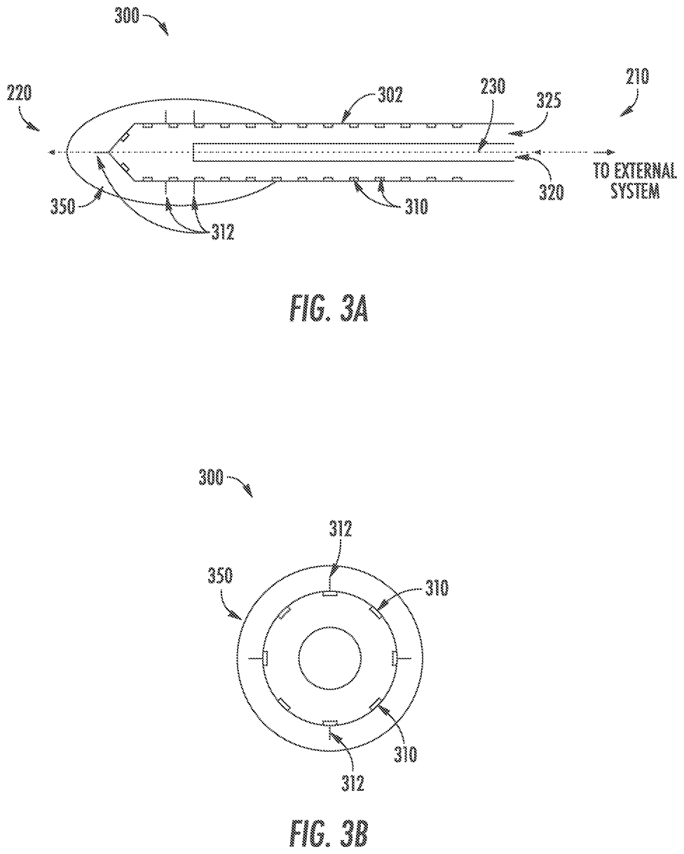

Referring now to FIGS. 3A and 3B, another example cryoablation probe 300 is shown. FIG. 3A is an axial cross section of the probe 300. FIG. 3B is a radial cross section of the probe 300. The probe 300 includes a tubular member 302 having a proximal end 210 and a distal end 220. The tubular member 302 has a probe tip 304 arranged at the distal end 220. A handle is arranged at the proximal end 210 of the tubular member 302. The probe 300 includes one or more energy elements 310. Alternatively or additionally, the probe 300 includes one or more sensor elements 312. The energy element(s) 310 and the sensor element(s) 312 are arranged along an axial direction 230 of the tubular member 302. In some implementations, the probe 300 includes both energy elements 310 and sensor elements 312 as shown in FIG. 3. In other implementations, the probe 300 may include only energy element(s) 310, for example an energy element configured to convert electrical energy to heat. In yet other implementations, the probe 300 may include only sensor element(s) 312, for example, a temperature sensor element that is configured to measure temperature in proximity to the tubular member 302. Optionally, as shown in FIG. 3, at least a portion of the sensor elements 312 protrude outward from the tubular member 302. This aids in measuring a local temperature in proximity to the probe 300. Optionally, the sensor elements 312 are retractable, e.g., the sensor elements 312 can extend outside the tubular member 302 and can be retracted inside the tubular member 302. For example, the sensor element 312 may be spring loaded in some implementations. The sensor element 312 may be retracted by default. When the user/operator chooses to deploy the sensor element 312, the user/operator engages the controls, and the springs force the sensor element 312 externally with respect to the tubular member 302. It should be understood that tissue surrounding the sensor element 312 is also displaced by force of the spring. When measurements are complete, the user/operator engages the controls to retract the sensor element 312. It should be understood that spring-loaded sensor elements 312 are provided only as an example. This disclosure contemplates that the sensor elements 312 may be deployed/retracted using alternative mechanisms.

Additionally, each of the energy elements 310 is configured to convert electrical energy to heat. For example, each of the energy elements 310 may be a resistive heating element. It should be understood that resistive heating elements are provided only as example energy elements 310. It should be understood that an energized energy element 310 generates heat, which causes temperature to increase and prevents formation of an ice block in vicinity to the energized energy element 310. In some implementations, each of the sensor elements 312 is configured to measure temperature. For example, each of the temperature sensors may be a thermistor or thermocouple. It should be understood that thermistors or thermocouples are provided only as example temperature sensors. As described herein, the probe 300 can include other types of sensors including, but not limited to, an inertial sensor (e.g., accelerometer, gyroscope, and/or magnetometer). Inertial sensors can be used for surgical navigation, e.g., determining the position and/or orientation of the probe 300 during a surgical procedure. In some implementations, the inertial sensor(s) can be integrated into to the probe 300. In other implementations, the inertial sensor(s) can be coupled to the probe 300.

As shown in FIG. 3, the probe 300 includes a plurality energy elements 310 arranged in a spaced apart relationship along the axial direction 230 of the tubular member 302. For example, the probe 300 can include between about 32 and about 64 energy elements. It should be understood that the number of energy elements 310 is provided only as an example. This disclosure contemplates having more or less energy elements 310 than provided as examples. Additionally, the probe 300 includes a plurality sensor elements 312 arranged in a spaced apart relationship along the axial direction 230 of the tubular member 302. For example, the probe 300 can include between about 32 and about 64 sensor elements. It should be understood that the number of sensor elements 312 is provided only as an example. This disclosure contemplates having more or less sensor elements 312 than provided as examples. Additionally, it should be understood that the number, spacing, and arrangement of the energy elements 310 and sensor elements 312 in FIG. 3 are provided only as an example. This disclosure contemplates providing a probe with different numbers, spacing, and/or arrangement of the energy elements 310 and sensor elements 312. This includes, but is not limited to, providing energy elements 310 and/or sensor elements 312 with even or uneven spacing between adjacent elements.

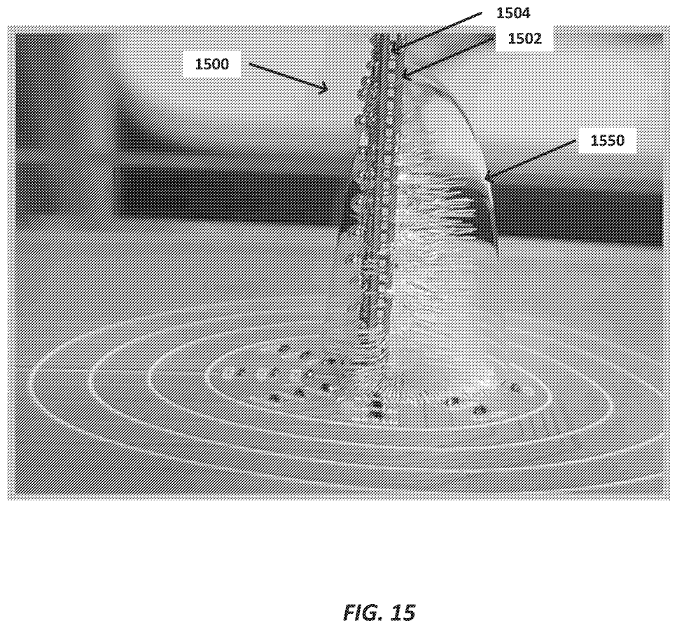

The probe 300 can include one or more compartments where fluids can flow and/or electronics can be embedded. For example, the probe 300 shown in FIG. 3 includes an internal compartment 320 and an external compartment 325. In FIG. 3, the electronics (e.g., energy elements 310 and sensor elements 312) are embedded in the external compartment 325 as described below. In other words, the energy elements 310 and the sensor elements 312 are arranged within the tubular member 302. In some implementations, the energy elements 310 and the sensor elements 312 are arranged on one or more flexible circuit boards, and the flexible circuit board(s) are embedded in the probe 300 (e.g., in the external compartment 325). As described above, the energy elements 310 may be resistive heating elements, and the sensor elements 312 may be thermistors or thermocouples. Such components can be mechanically mounted to and electrically connected via a flexible circuit board. Referring now to FIG. 15, a probe 1500 having four flexible circuit boards 1502, each having one or more energy elements and/or one or more sensor elements 1504, can be provided. The probe 1500 is placed in fluid filled container and operated to freeze fluid in the vicinity of the probe 1500. The ice block is labeled 1550. In FIG. 15, only one of the flexible circuit boards 1502 is labeled for simplicity. The four flexible circuit boards of the probe 1500 are placed adjacent to one another such that the elements 1504 are arranged around a circumference of the probe 1500. The length of each flexible circuit board extends the length of the probe 1500. It should be understood that the probe 1500, which includes a plurality of flexible circuit boards, shown in FIG. 15 is only an example. Referring again to FIG. 3, it should be understood that the location of the energy elements 310 and the sensor elements 312 in FIG. 3 (e.g., within the external compartment 325) is provided only as an example. This disclosure contemplates that the energy elements 310 and/or the sensor elements 312 can be located in any compartment of the probe 300 including, but not limited to, the internal compartment 320.

The probe 300 can also be operably coupled an external system such as a fluid expansion system, for example, as described above with regard to FIG. 1. Thermally conductive fluid is delivered to and circulated within the probe 300. Accordingly, the probe 300 includes a fluid channel arranged within the tubular member 302. In FIG. 3, the internal compartment 320 houses the fluid channel. This disclosure contemplates that the fluid channel can include inlet and/or return lines for circulating the thermally conductive fluid within the probe 300. The fluid channel is designed such that the thermally conductive fluid undergoes expansion within the tubular member 302, which causes temperature to decrease and formation of ice block(s) in the subject's body. It should be understood that the location of the fluid channel in FIG. 3 (e.g., within the internal compartment 320) is provided only as an example. This disclosure contemplates that the fluid channel can be located in any compartment of the probe 300 including, but not limited to, the external compartment 325.

Additionally, the probe 300 can be operably coupled an external system such as a controller, for example, as described above with regard to FIG. 1. The controller is configured to spatially and temporally control a cryoablation zone. Each of the energy element 310 and/or each of the sensor elements 312 is individually addressable. In other words, the controller is configured to selectively energize one or more of the energy elements 310. The controller is also configured to selectively obtain a respective measurement from one or more of the sensor elements 312. In some implementations, the controller is configured to address a plurality of energy elements 310 at the same time. For example, a first group of the energy elements 310 are arranged in a first axial region of the tubular member 302, and a second group of the energy elements 310 are arranged in a second axial region of the tubular member 302. In FIG. 3, the first group of energy elements 310 is within a cryozone 350 (e.g., the first axial region). This is the region where the probe 300 achieves extreme cold temperatures. The first group of energy elements 310, i.e., those in the cryozone 350, are not energized. The second group of energy elements 310 is outside the cryozone 350 (e.g., the second axial region). In contrast to the first group, the second group of energy elements 310 are energized, which prevents this region from achieving extreme cold temperatures. An ice block is therefore formed only in the cryozone 350. This allows the user to control the probe 300 to steer the cryozone 350, for example, to target specific tissue for ablation. The location of the cryozone 350 at the distal end 220 in FIG. 3 is provided only as an example. This disclosure contemplates that the cryozone 350 can be shifted proximally with respect to the probe 300. Additionally, it should be understood that the size, location, and/or number of cryozones in FIG. 3 are provided only as an example. Non-limiting examples are described in further detail below.

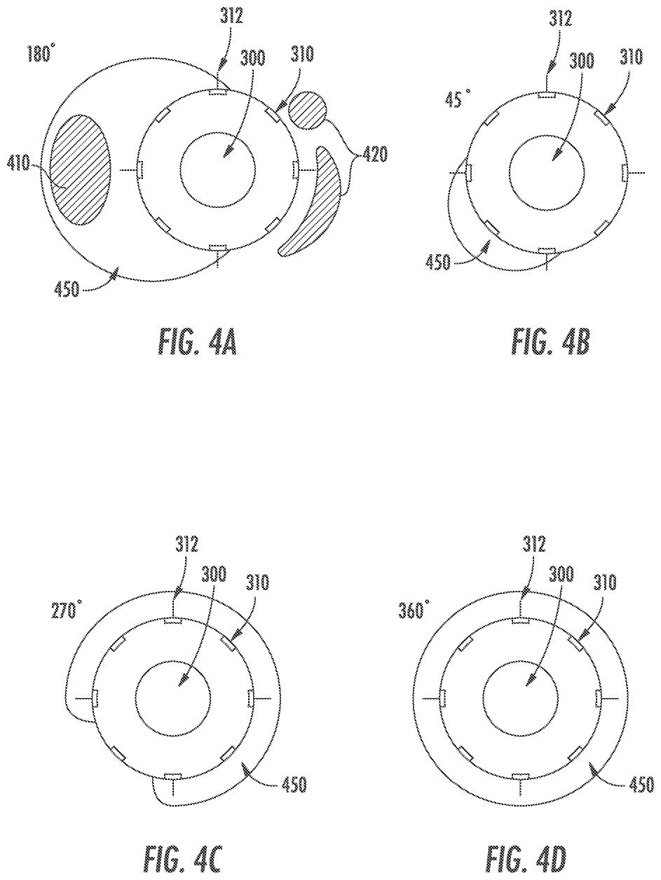

For example, in some implementations, a first group of the energy elements 310 are arranged in a first circumferential region of the tubular member 302 and a second group of the energy elements 310 are arranged in a second circumferential region of the tubular member 302. Referring now to FIGS. 4A-4D, radial cross sections of probes 300 controlled to achieve cryozones 450 of different angular sizes are shown. The probe 300 includes a plurality of energy elements 310 and a plurality of sensor elements 312. Similar to above, it should be understood that the number, spacing, and arrangement of the energy elements 310 and sensor elements 312 in FIGS. 4A-4D are provided only as an example. This disclosure contemplates providing a probe with different numbers, spacing, and/or arrangement of the energy elements 310 and sensor elements 312. This includes, but is not limited to, providing energy elements 310 and/or sensor elements 312 with even or uneven spacing between adjacent elements. As described herein, each of the energy element 310 and/or each of the sensor elements 312 is individually addressable such that the controller is configured to spatially and temporally control a cryoablation zone.

In FIG. 4A, the first group of energy elements 310 is within the cryozone 450 (e.g., the first circumferential region). This is the region where the probe 300 achieves extreme cold temperatures. The first group of energy elements 310, i.e., those in the cryozone 450, are not energized. This 180° region in the circumferential direction of the probe 300 is in proximity to target tissue 410. The second group of energy elements 310 is outside the cryozone 450 (e.g., the second circumferential region). In contrast to the first group, the second group of energy elements 310 are energized, which prevents this region from achieving extreme cold temperatures. This 180° region in the circumferential direction of the probe 300 is in proximity to non-target tissue 420. An ice block is therefore formed only in the cryozone 450, which prevents non-target tissue 420 from exposure to extreme cold temperature (and possible damage and/or destruction).

In FIG. 4B, the first group of energy elements 310 is within the cryozone 450 (e.g., the first circumferential region). The first group of energy elements 310, i.e., those in the cryozone 450, are not energized. This 45° region in the circumferential direction of the probe 300 may be in proximity to target tissue (not shown). The second group of energy elements 310 is outside the cryozone 450 (e.g., the second circumferential region). In contrast to the first group, the second group of energy elements 310 are energized. This 315° region in the circumferential direction of the probe 300 may be in proximity to non-target tissue (not shown). An ice block is therefore formed only in the cryozone 450, which prevents non-target tissue from exposure to extreme cold temperature (and possible damage and/or destruction).

In FIG. 4C, the first group of energy elements 310 is within the cryozone 450 (e.g., the first circumferential region). The first group of energy elements 310, i.e., those in the cryozone 450, are not energized. This 270° region in the circumferential direction of the probe 300 may be in proximity to target tissue (not shown). The second group of energy elements 310 is outside the cryozone 450 (e.g., the second circumferential region). In contrast to the first group, the second group of energy elements 310 are energized. This 90° region in the circumferential direction of the probe 300 may be in proximity to non-target tissue (not shown). An ice block is therefore formed only in the cryozone 450, which prevents non-target tissue from exposure to extreme cold temperature (and possible damage and/or destruction).

In FIG. 4D, none of the energy elements 310 are energized, and the cryozone 450 is a 360° region in the circumferential direction of the probe 300. It should be understood that the size (e.g., angular extent) and/or location of the cryozone 450 in FIGS. 4A-4D are provided only as examples. As described herein, the energy elements 310 are individually addressable such that the user can selectively energize one or more of the energy elements 310 to steer the cryozone 450, for example, to achieve ice block formation in a desired region. This disclosure contemplates that energy elements 310 can be addressed to achieve a cryozone of variable sizes, in some implementations greater than or equal to about 30° in the circumferential direction of the probe 300. Additionally, the location of the center of the cryozone 450 is not intended to be limited (e.g., the center may be located 0-360° relative).

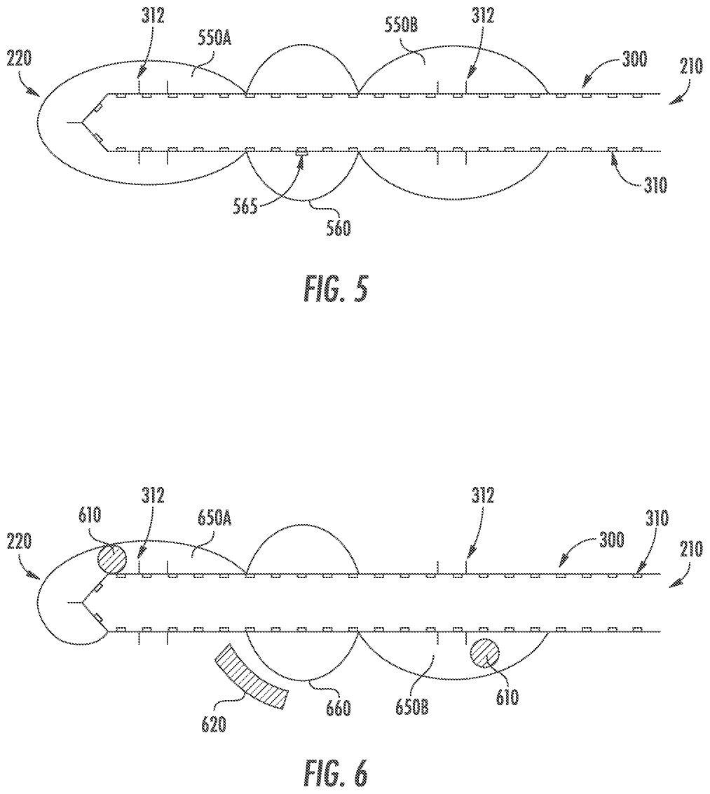

Referring again to FIG. 3, in some implementations, the probe 300 can be controlled to create a plurality of distinct cryozones. In other words, the single cryozone 350 shown in FIG. 3 is provided only as an example. This disclosure contemplates the probe 300 can be controlled to form multiple distinct cryozones, each cryozone located in a different spatial location (e.g., axially and/or circumferentially with respect to the probe 300). For example, referring now to FIG. 5, an axial cross section of probe 300 controlled to achieve a plurality of cryozones 550A and 550B is shown. The probe 300 includes a plurality of energy elements 310 and a plurality of sensor elements 312. Similar to above, it should be understood that the number, spacing, and arrangement of the energy elements 310 and sensor elements 312 in FIG. 5 are provided only as an example. This disclosure contemplates providing a probe with different numbers, spacing, and/or arrangement of the energy elements 310 and sensor elements 312. As described herein, each of the energy element 310 and/or each of the sensor elements 312 is individually addressable such that the controller is configured to spatially and temporally control a cryoablation zone.

As shown in FIG. 5, a first cryozone 550A is formed at the distal end 220 and a second cryozone 550B is formed proximally with respect to the first cryozone 550A. It should be understood that the sizes and locations of the cryozones 550A and 550B in FIG. 5 are provided only as examples. Additionally, the number of cryozones (two in FIG. 5) is also provided only as an example. The cryozones 550A and 550B are formed by not energizing the energy elements 310 in those regions. In contrast, the energy elements 310 outside of the cryozones are energized, which prevents ice block formation outside of the cryozones 550A and 550B.

Optionally, as shown in FIG. 5, the probe 300 can further include an elastic layer 560 (e.g., a balloon). The elastic layer 560 is configured to expand when fluid such as air or saline is introduced. The elastic layer 560, when filled, can isolate the cryozones 550A and 550B from each other. Optionally, the probe 300 can further include a strain gauge 565, which detects pressure inside the balloon. It should be understood that this information can be used to control inflation/deflation of the balloon.

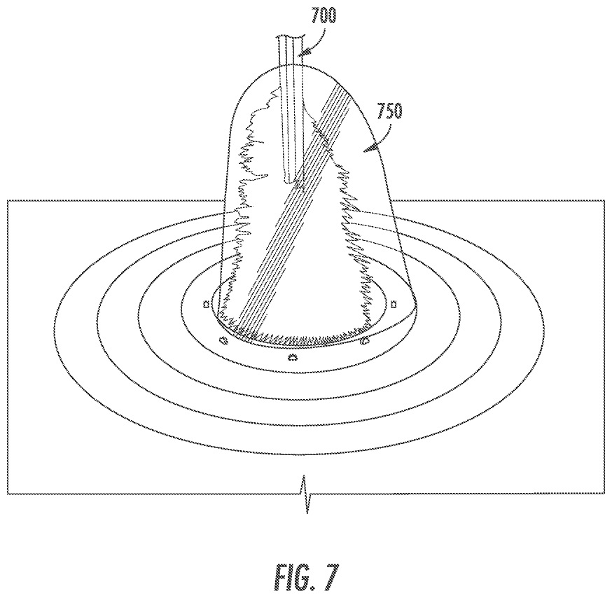

Referring now to FIG. 6, an axial cross section of probe 300 controlled to achieve a plurality of cryozones is shown. The probe 300 includes a plurality of energy elements 310 and a plurality of sensor elements 312. Similar to above, it should be understood that the number, spacing, and arrangement of the energy elements 310 and sensor elements 312 in FIG. 6 are provided only as an example. This disclosure contemplates providing a probe with different numbers, spacing, and/or arrangement of the energy elements 310 and sensor elements 312. As described herein, each of the energy element 310 and/or each of the sensor elements 312 is individually addressable such that the controller is configured to spatially and temporally control a cryoablation zone.

As shown in FIG. 6, two distinct cryozones are formed. A first cryozone 650A is formed at the distal end 220 and a second cryozone 650B is formed proximally with respect to the first cryozone 650A. In FIG. 6, the cryozones are spaced apart axially and also arranged in different circumferential regions with respect to the probe 300. For example, the first cryozone 650A and the second cryozone 650B are formed in proximity to target tissues 610 (e.g., target nerves), each target tissue being located in a different region circumferentially with respect to the probe 300. This can be achieved by energizing the appropriate energy element 310 to prevent ice block formation in proximity to non-target tissue 620. It should be understood that the sizes and locations of the cryozones 650 in FIG. 6 are provided only as examples. Additionally, the number of cryozones (two in FIG. 6) is also provided only as an example

Additionally, as shown in FIG. 6, the probe 300 can optionally further include an elastic layer 660 (e.g., a balloon). The elastic layer 660 is configured to expand when fluid such as air or saline is introduced. The elastic layer 660 can isolate the cryozones 650A and 650B from each other when deployed as a balloon.

Referring again to FIG. 3, the probe 300 includes a plurality of sensor elements 312. As described herein, the sensor elements 312 can be temperature sensors. In other words, sensor elements 312 such as temperature sensors can be integrated into the probe 300. It should be understood that temperature sensors can be used to measure temperature in proximity to the probe 300. For example, the temperature sensors can be used to measure local tissue temperature in proximity to the probe 300. Local tissue temperature is measured at a distance from the probe by temperature sensor(s) integrated in the probe 300. In some implementations, the temperature sensors measure local tissue temperature a distance about 2 millimeters (mm) from the probe 300. In other implementations, the temperature sensors measure local tissue temperature a distance greater than 2 mm from the probe 300, for example, about 3, 4, 5, . . . 10 mm. In other implementations, the temperature sensors measure local tissue temperature a distance greater than 10 mm from the probe 300, for example, about 15, 20, 25, . . . 1 centimeter (cm). As described herein, the number and arrangement of the sensor elements 312 in the figures are provided only as examples. For example, in FIG. 3A, sensor elements 312 are arranged at the tip of the probe 300, as well as near the distal end 220. In FIGS. 3B and 4A-4C, sensor elements 312 are arranged around the circumference of the probe 300 (e.g., spaced apart, every 90°). In FIGS. 5 and 6, sensor elements 312 are arranged at the tip of the probe 300, as well as in two regions along the axial direction of the probe 300. This disclosure contemplates that the number and arrangement of sensor elements 312 can be selected to provide a desired sensing resolution. The sensor elements 312 can be used to monitor conditions (e.g., temperature) in proximity to the probe 300 in real-time.

Referring again to FIG. 1, the cryoablation system 100 includes the cryoablation probe 102, the fluid expansion system 104, and the controller 106. This disclosure contemplates that the cryoablation probe 102 can be any one of the probes described with respect to FIGS. 1-8A and 15. The cryoablation probe 102 can be used to perform a percutaneous cryoablation procedure on a target tissue. In some implementations, the target tissue is a nerve. In other implementations, the target tissue is a tumor, ganglia, or adipose tissue. After inserting the cryoablation probe 102 into the subject, the cryoablation probe 102 is operated to create a cryozone, e.g., a region where ice forms in a subject's body as a result of the low temperatures of the probe 102. The controller 106 is configured to spatially and temporally control the cryoablation zone, for example, the cryozone of FIGS. 3A and 3B. In some implementations, the controller 106 is configured to spatially and temporally control a plurality of cryoablation zones, e.g., the cryozones of FIGS. 5 and 6. The controller 106 spatially and temporally controls the cryozone(s) by individually addressing and energizing one or more energy elements (e.g., energy elements 310 of FIGS. 3A-6).

In some implementations, the controller 106 adjusts the size and/or shape of the cryozone(s) by individually addressing and energizing energy elements. In some implementations, the controller 106 selects an angular region for the cryozone(s). For example, the angular region may be equal to or greater than about a 30° sector in a circumferential direction. FIGS. 4A-4D illustrate 180°, 45°, 270°, and 360° cryozones, respectively. In some implementations, the controller 106 steers the cryozone(s). For example, the cryozone(s) can be rotated in a circumferential direction. Optionally, a direction of rotation can be switched. This disclosure contemplates that the operations described above can optionally be performed in real time. Alternatively or additionally, the operations described above can be initiated by a user/operator or by pre-programmed control algorithms.

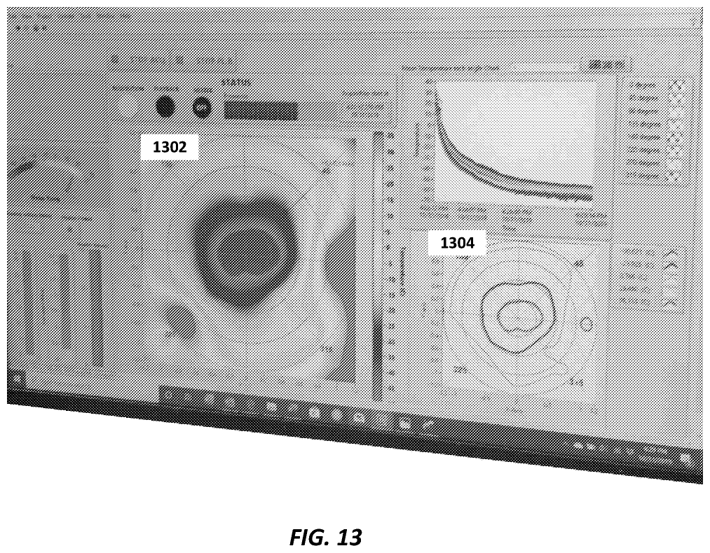

The controller 106 also receives a measurement detected by at least one of the sensor elements (e.g., sensor elements 312 of FIGS. 3A-6). In some implementations, the sensor element(s) are temperature sensors. The controller 106 can optionally provide real-time feedback based on the detected measurements. In some implementations, the real-time feedback is local tissue temperature in proximity to the probe 102. Optionally, the real-time feedback is at least one of a visible, audible, or tactile alarm. In some implementations, the system 100 further includes a display device, and the real-time feedback is displayed on the display device. An example user interface for display on the display device is shown in FIG. 13. The user interface may include a heat plot 1302 and a heat contour plot 1304. It should be understood that the temperatures displayed in the heat plot 1302 and/or the contour plot 1304 can be measured by at least one of the sensor elements (e.g., sensor elements 312 of FIGS. 3A-6). A user/operator can use the user interface of FIG. 13 for controlling the probe during the procedure. For example, the user/operator can use the information displayed via such user interface to understand when the probe achieves the target treatment temperature, time at target temperature, and/or size and shape of the cryozone. It should also be understood that FIG. 13 is provided only as an example. This disclosure contemplates that a user interface can include the same and/or different information, as well as display information in different form than as shown in FIG. 13. Additionally, this disclosure contemplates that measured temperatures (e.g., such as those shown in FIG. 13) may optionally be displayed along with surgical guidance images (e.g., CT, MRI, ultrasound). This disclosure also contemplates that visualization of anatomical target, probe placement, ongoing ablation, and temperature are in real time. The real-time knowledge of tissue temperature at a given distance from the probe allows for precise, timed, uniform decrease of temperature across the targeted tissue (e.g., nerve). Visualization of probe placement can be under direct image guidance with tracking software which will allow real-time precision placements. Optionally, the real-time feedback (e.g., local tissue temperature in proximity to the probe 102) can be used to control the probe 102, for example, to destroy the tissue and/or treat a condition. Alternatively or additionally, the controller 106 individually addresses and energizes one or more energy elements based on the real-time feedback. In other words, the controller 106 can be configured to adjust the size and/or shape, location, angular extent, and/or steer cryozone(s) automatically in response to the detected measurements.

In some implementations, the controller 106 optionally receives a measurement detected by one or more inertial sensors, which can be integrated with or coupled to the probe 102. Each inertial sensor can include one or more accelerometers, one or more gyroscopes, one or more magnetometers, or combinations thereof. Inertial sensor(s) can be used for surgical navigation, e.g., determining the position and/or orientation of the probe 102 during a surgical procedure. For example, in some implementations, the probe 102 can be housed in sterile housing, and sterile housing can be adhered to a subject's body. Surgical images, for example a CT scan, can be captured of the subject with both the surgical site (which includes the anatomical target) and sterile housing in the field of view. The sterile housing and the probe 102 can include one or more fiducial markers (e.g., beads or other elements) that are visible in the CT scan. Such fiducial markers captured in the CT scan can be used to align the probe 102 and the sterile housing. Measurements obtained by the inertial sensor(s) can then be used to track the position and/orientation of the probe 102 with respect to the sterile housing during the surgical procedure. Additionally, the position and/or orientation of the probe 102 can be displayed for the user/operator relative to the CT scan, which includes the anatomical target. It should be understood that this display and information can be provided to the user/operator in real-time during the surgical procedure.

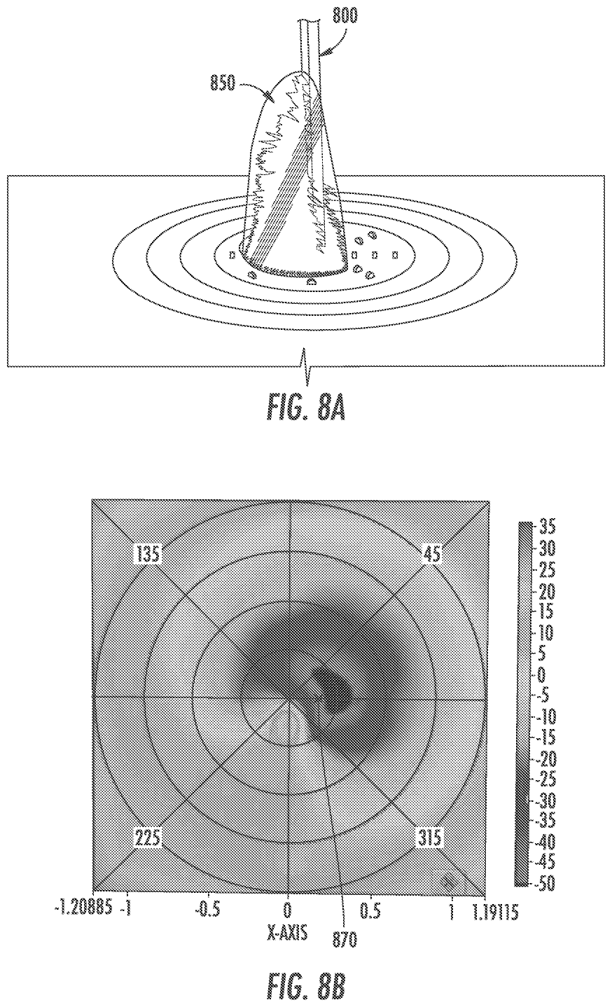

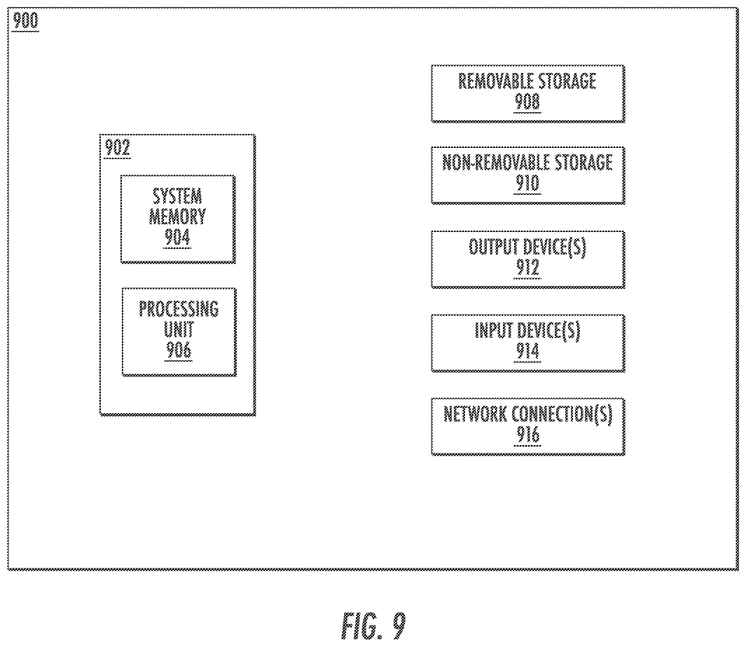

Referring now to FIGS. 7 and 8A, images of ice blocks formed by example cryoablation probes are shown. The probes of FIGS. 7 and 8A were placed in fluid filled container and operated to freeze fluid in the vicinity of the probes. FIG. 7 illustrates an ice block 750 formed 360° around the cryoablation probe 700. The cryoablation probe 700 includes a plurality of energy elements that facilitate thermo-electric control of the ice block 750. FIG. 8A illustrates an ice block 850 formed around a 180° sector of a probe 800. The cryoablation probe 800 includes a plurality of energy elements that facilitate thermo-electric control of the ice block 850. In particular, a subset of the energy elements are energized to prevent ice block formation in a 180° sector of the probe 800. FIG. 8B is a graph illustrating the local temperatures in proximity to the probe 800 of FIG. 8A. The local temperatures shown in FIG. 8B were obtained by bench measurements at the base of the fluid filled container. The probe is centered at point 870 in FIG. 8B.

Example Computing Device

It should be appreciated that the logical operations described herein with respect to the various figures may be implemented (1) as a sequence of computer implemented acts or program modules (i.e., software) running on a computing device (e.g., the computing device described in FIG. 9), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 9, an example computing device 900 upon which the methods described herein may be implemented is illustrated. It should be understood that the example computing device 900 is only one example of a suitable computing environment upon which the methods described herein may be implemented. Optionally, the computing device 900 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 900 typically includes at least one processing unit 906 and system memory 904. Depending on the exact configuration and type of computing device, system memory 904 may be volatile (such as random access memory (RAM)), non-volatile (such as read-only memory (ROM), flash memory, etc.), or some combination of the two. This most basic configuration is illustrated in FIG. 9 by dashed line 902. The processing unit 906 may be a standard programmable processor that performs arithmetic and logic operations necessary for operation of the computing device 900. The computing device 900 may also include a bus or other communication mechanism for communicating information among various components of the computing device 900.

Computing device 900 may have additional features/functionality. For example, computing device 900 may include additional storage such as removable storage 908 and non-removable storage 910 including, but not limited to, magnetic or optical disks or tapes. Computing device 900 may also contain network connection(s) 916 that allow the device to communicate with other devices. Computing device 900 may also have input device(s) 914 such as a keyboard, mouse, touch screen, etc. Output device(s) 912 such as a display, speakers, printer, etc. may also be included. The additional devices may be connected to the bus in order to facilitate communication of data among the components of the computing device 900. All these devices are well known in the art and need not be discussed at length here.

The processing unit 906 may be configured to execute program code encoded in tangible, computer-readable media. Tangible, computer-readable media refers to any media that is capable of providing data that causes the computing device 900 (i.e., a machine) to operate in a particular fashion. Various computer-readable media may be utilized to provide instructions to the processing unit 906 for execution. Example tangible, computer-readable media may include, but is not limited to, volatile media, non-volatile media, removable media and non-removable media implemented in any method or technology for storage of information such as computer readable instructions, data structures, program modules or other data. System memory 904, removable storage 908, and non-removable storage 910 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 906 may execute program code stored in the system memory 904. For example, the bus may carry data to the system memory 904, from which the processing unit 906 receives and executes instructions. The data received by the system memory 904 may optionally be stored on the removable storage 908 or the non-removable storage 910 before or after execution by the processing unit 906.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Example Methods

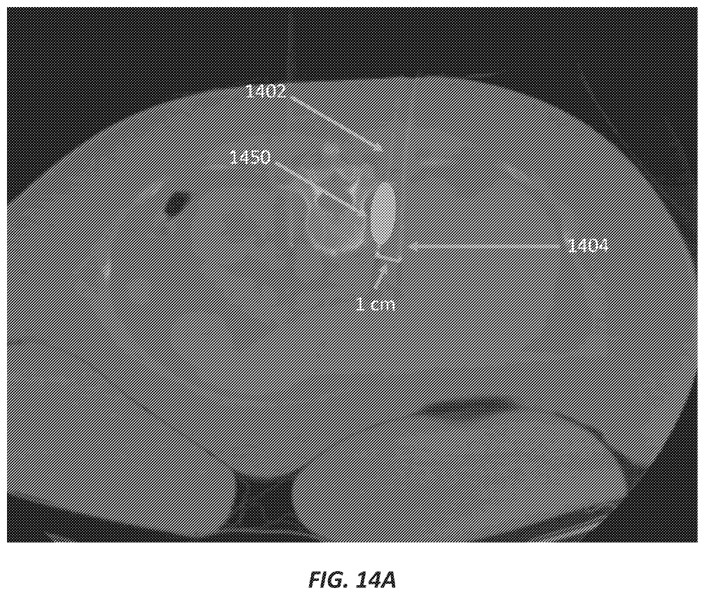

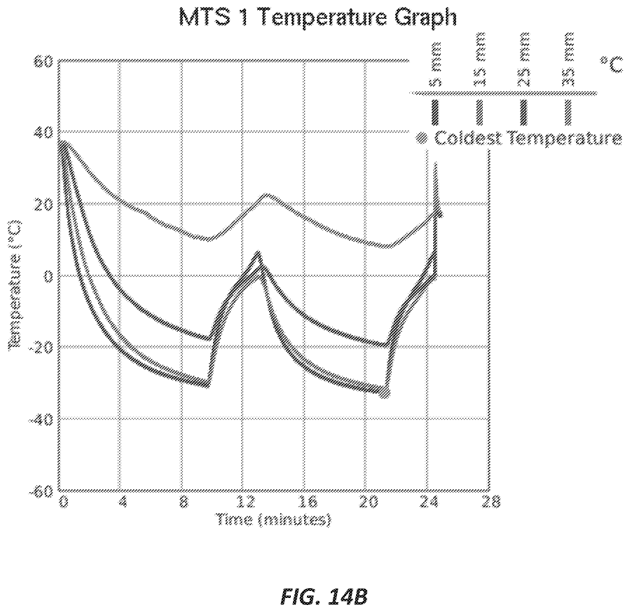

Referring now to FIGS. 14A and 14B, a cryoablation procedure using a conventional cryoablation probe is described. FIG. 14A is a CT image showing both a conventional cryoablation probe 1402 and a temperature sensing probe 1404 inserted into a patient's anatomy during the procedure. The cryoablation probe 1402 is located about 1 cm from the temperature sensing probe 1404. The ice cryozone is labeled 1450 in FIG. 14A. As described above, the cryoablation probe 1402 and the temperature sensing probe 1404 are separate probes, i.e., each requires its own incision. Thus, the cryoablation probe 1402 and the temperature sensing probe 1404 are spaced apart at the surgical site such that the temperature measurements captured by the temperature sensing probe 1404 only approximate temperature at the anatomical target, which is closer to the cryoablation probe 1402. It can be difficult, if not impossible, to place the cryoablation probe 1402 and the temperature sensing probe 1404 any closer to each other (e.g., less than 1 cm). The temperature sensing probe 1404 measures temperature at a plurality of points along its longitudinal axis (e.g., 5 mm, 15 mm, 25 mm, 35 mm) extending from its distal end. FIG. 14B is a graph illustrating tissue temperatures over time measured by the temperature sensing probe 1404 of FIG. 14A. The graph includes plots for the respective temperatures over time measured at 5 mm, 15 mm, 25 mm, and 35 mm along the longitudinal axis of the temperature sensing probe. As shown in FIG. 14B, the coldest (i.e., lowest) temperature achieved is about −30° C. at about 22 minutes measured at the 5 and 15 mm locations along the temperature sensing probe. It should be understood that −30° C. and/or the exposure time at this temperature may be insufficient to achieve the desired physiological effects for cryoablation of nerves. Additionally, it should be understood that temperature measured by the temperature sensing probe only approximate conditions in proximity to the cryoablation probe.

Example methods for cryoneurolysis and cryoablation are described herein. These methods can include performing a cryovagotomy, cryosplanchnicetomy, or cryoneurolysis or cryoablation of another nerve. Additionally, the methods described can be used to treat conditions including, but not limited to, a metabolic syndrome, type 2 diabetes, hypertension, obesity, sexual dysfunction, chronic pain, phantom limb pain, or a tumor. It should be understood that the cryoablation probes and/or systems described with regard to FIGS. 1-8A and 15 can be used to perform the methods described herein. The cryoablation probes and/or systems shown in FIGS. 1-8A and 15 can improve conventional processes for at least the following reasons. The cryoablation probes and/or systems shown in FIGS. 1-8A and 15 facilitate spatial and temporal control of one or more cryozones. This is optionally accomplished in real time during the surgical procedure. Alternatively or additionally, the cryoablation probes and/or systems shown in FIGS. 1-8A and 15 provide the user/operator with real-time feedback about local tissue temperature. Unlike conventional cryoablation probes, the local tissue temperatures are measured by sensors integrated into the cryoablation probe (i.e., as opposed to measured by an additional temperature sensing probe). Such real-time feedback allows the user/operator to better understand (and optionally control) the target treatment temperature achieved by the cryoablation probe and/or exposure time. As described herein, this allows the user/operator to control the treatment temperature and/or exposure time according to the particular procedure to achieve the desired result. Additionally, this allows the user/operator to minimize or eliminate the risk of damaging non-target tissue.

An example method is also described herein. The method can include using a cryoablation probe (e.g., any one of the probes shown in FIGS. 1-8A and 15) to perform a percutaneous cryoablation procedure on a target tissue, and receiving real-time feedback of local temperature in proximity to the cryoablation probe. The method can also include using the real-time feedback of local temperature in proximity to the cryoablation probe to control the cryoablation probe and destroy the tissue.

In some implementations, the target tissue is a nerve. This disclosure contemplates using the real-time feedback of local temperature to control the treatment temperature and/or the exposure time. For example, in some implementations, the step of using the real-time feedback of local temperature includes controlling the cryoablation probe to achieve Wallerian degeneration of the nerve. Wallerian degeneration of the nerve is achieved by controlling the local temperature to achieve a target temperature and/or an amount of time at the target temperature. In some implementations, the step of using the real-time feedback of local temperature includes controlling the cryoablation probe to induce a Sunderland 2 injury. It should be understood that the achieving Wallerian degeneration or inducing Sunderland 2 injury are provided only as examples. Some treatments may require higher or lower treatment temperatures and/or longer or shorter treatment times, including inducement of different Sunderland injuries. In other implementations, the target tissue is a tumor, ganglia, or adipose tissue. Tumor, ganglia, and adipose tissue can be destroyed by ice formation in fluid outside the target cells (which results in dehydration), ice formation inside the target cells, and/or swelling/shrinking of the target cells caused by ice formation inside the target cells. Additionally, the step of using the real-time feedback of local temperature includes controlling the cryoablation probe to achieve the desired temperature needed to destroy the target cells.

Another example method is described herein. The method includes using a cryoablation probe (e.g., any one of the probes shown in FIGS. 1-8A and 15) to perform a percutaneous cryoablation procedure on a target tissue, and receiving real-time feedback of local temperature in proximity to the cryoablation probe. The method also includes using the real-time feedback of local temperature in proximity to the cryoablation probe to control the cryoablation probe and treat a condition. The condition may be a metabolic syndrome, type 2 diabetes, hypertension, obesity, sexual dysfunction, chronic pain, phantom limb pain, or a tumor.

Example procedures and/or treatments are described below. This disclosure contemplates that the cryoablation probes and/or systems described with regard to FIGS. 1-8A and 15 can be used to perform these procedures and/or treatments.

Cryoneurolysis

The devices and systems described herein can be used for cryoneurolysis. Such procedures attempt to ablate specific anatomical tissues to treat a variety of chronic disorders. The target anatomical tissues can be of various geometries, be at various locations relative to other organs, and be under significantly different thermal stresses depending upon the patient's body composition. To achieve targeted, complete, and effective cryoneurolysis (or cryoablation) the probe geometries can be designed to enable appropriate contact with the tissues. In many cases, these geometries can be complex with structures that are not easy to pass through the skin, organs, and nearby tissues to reach the target.

As described herein, a method for deploying complex geometry cryoneurolysis (or cryoablation) devices/probes to contact target tissues of various geometries (e.g., nerves, ganglia, tumors) is provided. The core of the technology is a coaxial insertion system which consists of a guide tube and a removable/inner needle. The coaxial system is used to puncture through the skin and navigate to the target tissue location. Once at the target location, the guide tube is affixed to the patient skin surface with a biocompatible and temporary adhesive and the inner needle is removed leaving a hollow guide tube. The cryoneurolysis (or cryoablation) probe is placed through the tube, enabling quick and accurate placement of the probe at the target location.

Cryoablation of peripheral nerves and/or ganglia results in complete and safe treatment of a myriad of chronic diseases (e.g., diabetes, obesity, hypertension, premature ejaculation). Although the technology has been around for years, the parameters and protocols associated with achieving a safe, effective, and complete ablation are unclear and typically chosen at random. The ability to target complex structures such as peripheral nerves or ganglia remains elusive to even most experienced clinicians performing the procedure. Furthermore, the ability to regulate the size, timing, and tissue that is targeted by the cryoablation is currently unavailable. Existing cryoablation probes are simply long needles. The devices, systems, and methods described herein pave the path for cryoablation of nerves by treating the following unmet needs. The devices, systems, and methods described herein allow for spatiotemporal control of temperature gradients. The probes may be capable of creating probe-tissue temperatures as low as −80° C. Cooling may be provided by either gas, electrical, thermochemical, or a combination of approaches. Diameter of the probe may be up to 1 centimeter (cm). The probe may include at least 1 set of electrical contacts to measure electrical impedance. The probe may include at least 1 set of electrical contacts to measure physiological signals from the target structure. The probe may include at least 1 sensor for measurement of thermal, electrical, mechanical, and anatomical properties of the target/contact and surrounding tissue, such as temperature, blood flow. The probe may match geometry to the target tissue (circular for nerves, planar for organs or ganglia, etc.)

During the onset of cryoneurolysis, fibers/axons in nerve bundles can intermittently be activated resulting in perception of pain by the patient. Furthermore, patients may also feel a brief period of post-operative pain as well. The devices, systems, and methods described herein provide an approach to treating pre- and post-operative pain associated with cryoneurolysis. The probe may include gel or hydrogel bioactive coating that can deliver bioactive compounds (drugs, agents, etc.) for analgesia. The probe may include gel or hydrogel bioactive coating that can focus the temperature gradient toward the target tissue. The anesthetics/analgesics can include, but are not limited to, fast-acting, sustained release bupivacaine, lidocaine, etc. The gel and hydrogels can be applied in varying thicknesses based upon the anatomy/size of the target structure. The hydrogels can contain any base material—such as PEG, PEG-heparin, or other biocompatible materials.

The devices, systems, and methods described herein provide the ability to identify, target, and reach nerve locations. As described above, placement of ablation probes near the correct anatomical target is challenging and if placed incorrectly, can result in damage and long-term consequences to the patient. Furthermore, insertion of the probe is conducted blindly without being able to directly observe vessels or other structures in the path to the target tissue. Existing ablation probes, either for cryoablation of RF or microwave, are simply long needles. Placement of these probes to target nerves or other tissues in the body can be challenging due to no direct visual of the object in front of the probe once the probe enters the body. The devices, systems, and methods described herein can optionally be paired with computer vision, machine learning, and image processing algorithms and techniques to tell the user/operator where the target is and how to get there, thereby increasing the efficiency, accuracy, and speed of the procedures. Systems may use computed tomography (CT) images taken before the procedure and provide a 3D visual for the physician to observe all tissues in the target region and registering the location of the patient to a nearby tracking camera. Image guidance may be applied to ultrasound or other imaging modalities. Machine learning algorithms may identify the type of tissue and structure in the 3D volume and provide a suggested entry point, angle, trajectory, and depth for insertion of the probe. Fiducial marker(s) may be placed on the probes to track the motion of the probe by the physician. The inserted probe's exact location may be displayed in the 3D visual generated from the CT image. Suggested parameters for ablation may be suggested based upon the geometry extrapolated from the CT image. The probe may include markings that enable computer vision based computer-readable identifiers (e.g., fiducials). The probe may be coated with materials that enable visualization under image guidance such as fluoroscopy, CT, ultrasound, etc. Physical markers that are CT (or image modality specific) opaque can be used to track tip or end of probe.

During the cryoablation procedure, patients are in the supine or prone positions and the physician inserts the cryoablation device to the target tissue location under image guidance. Once placed, the probe ideally maintains its position and contact with the target tissue. However physiological (and non-physiological) artifacts—such as respiratory motion, muscle contractions, and patient motion—lead to motion at the device-target tissue interface. These artifacts can contribute to impartial ablations, damage to nearby tissue (off-target effects), and oscillations in the temperature gradients desired to obtain a complete and successful cryoablation. The devices, systems, and methods described herein address the above issues. For example, the systems use a multi-component mechanism to maintain a consistent contact with the target tissue during the procedure. The probe may include a probe tip, a mechanical damper, and the probe handle and connector. The tip and probe handle may be connected by a mechanical damper. The damper may be a self-actuating mechanical component such as a mechanical spring, hydraulic damper, dashpot or other system that enables maintenance of consistent and reliable contact with the target tissue and minimizes motion of the probe during motion artifacts.

The target anatomical tissues can be of various geometries, be at various locations relative to other organs, and be under significantly different thermal stresses depending upon the patient's body composition. To achieve targeted, complete, and effective cryoneurolysis (or cryoablation), the probe geometries may be designed to enable appropriate contact with the tissues. The devices, systems, and methods described herein address the above issues. The geometry of the probe may be designed to match the target tissue (e.g., curved surfaces on probes as opposed to simple needles). A cryoneurolysis (or cryoablation) probe tip may be a needle or other complex geometry such as a semi-circle, triangular, or rectangular. Spatially arranged features within the needle may be used to control the direction and profile of the temperature gradient, thus enabling control of which tissues are ablated (which tissues are not). Sensor fusion technology (sensing electrical, mechanical, and thermal properties through the probe) may be used to provide direct feedback about localization of the target nerve or ganglia. The system may provide a suggested protocol to use for cryoablating the nerve. The system may provide feedback on whether the target has been completely cryoablated to ensure therapeutic benefit.

In some implementations, probe geometries can be complex with structures that are not easy to pass through the skin, organs, and nearby tissues to reach the target. The devices, systems, and methods described herein provide a method for deploying complex geometry cryoneurolysis devices/probes to contact target tissues of various geometries (e.g., nerves, ganglia, tumors). The devices, systems, and methods described herein address the above issues by providing: co-axial insertion system for deployment of probe to target site, guide tube and probe geometry can be a cylinder or other polygonal (e.g., hexagon, pentagon, etc.) structure, guide tube or probe can be made of a metallic or non-metallic material, guide tube which aides in stabilization, targeting, and initial deployment of the probe, coaxial system may consist of sensors for measurement of tissue properties.

Nerves are complex structures with multiple different types of axons/fibers (myelinated, unmyelinated) which carry many different types of signals (motor, sensory, pain, etc.). In many conditions, ablation of an entire nerve is not desired or ideal and could lead to significant side effects. Cryoablation therapy can provide for fiber-type specific cryoablation when the parameters are chosen for optimal cooling per fiber type. In some implementations, the devices, systems, and methods described herein can be used to ablate myelinated or motor fibers only. In other implementations, the devices, systems, and methods described herein can be used to ablate myelinated and unmyelinated (or motor and sensor) fibers altogether.

The devices, systems, and methods described herein, which use real-time feedback of local temperature, can be used to control the probe to achieve Wallerian degeneration of a nerve. As described herein, Wallerian degeneration is a mechanism of effect of cryoablation for treatment of conditions related to nerves. Sunderland 2 injury results in predictable Wallerian degeneration with subsequent axonal regeneration. Sunderland 2 injury has been correlated with nerve exposure to temperatures ranging from −20° to −100° Celsius. Partial ablation of a nerve results in unwanted clinical sequela, including pain, allodynia, and/or symptom worsening. Partial ablation also precludes the desired clinical effect. For example, if the desired clinical effect is nerve repair through regeneration or nerve degeneration in order to decrease conduction, partial ablation will leave axons intact and preclude the desired clinical effect by leaving damaged nerves in place, preserving function, or even damaging the nerve. Several studies of cryoneurolysis have reported allodynia, partial effect, or symptom worsening following cryoablation of a targeted nerve. The explanation for these symptoms is partial or under-ablation of the target nerve, resulting in a Sunderland 1 or mixed Sunderland 1/2 injury. The desired injury is not instantaneous and requires continued exposure to cold for a specific amount of time, depending on the diameter and orientation of the targeted nerve. Complete ablation of a targeted nerve depends on uniform temperature drop across the nerve in the range of −20° to −100° Celsius, which is not obtained with the currently reported times of exposure using conventional probes because of, a) inability to measure the in vivo temperature during the ablation, b) varying effects of tissue type, tissue depth, and adjacent blood flow on the temperature of the ablation zone and targeted nerve, and c) diameter and orientation of the nerve.

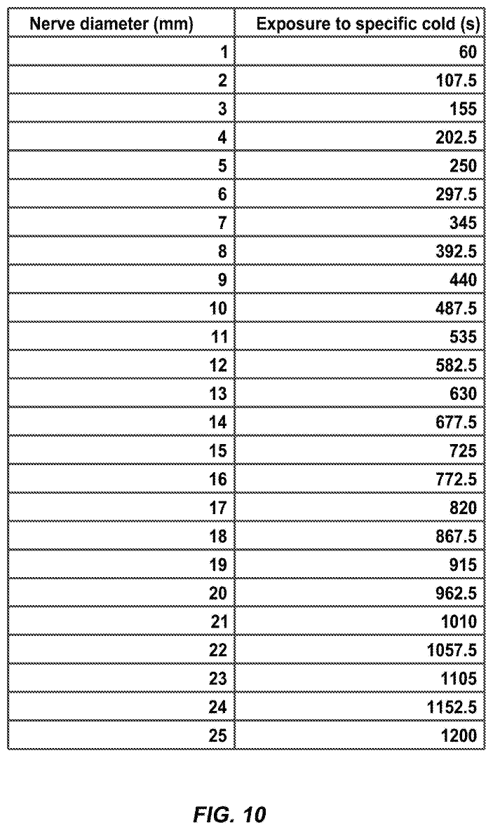

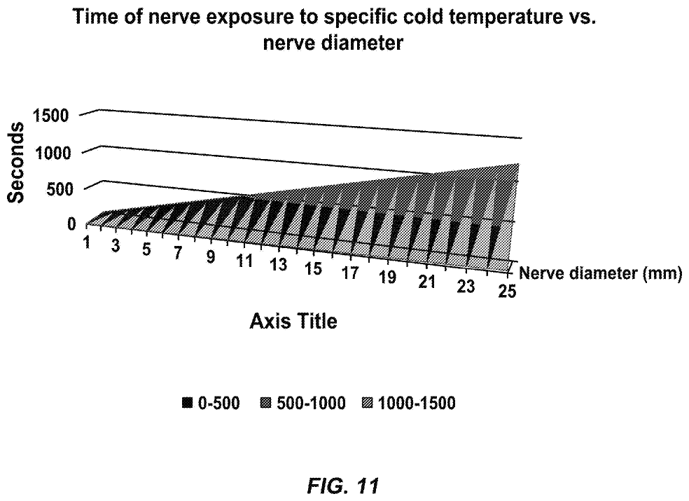

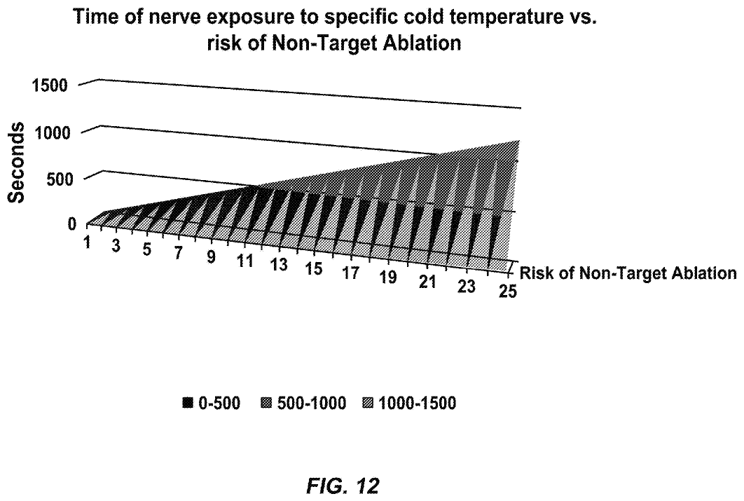

The necessary time of exposure to cold in the −20° to −100° Celsius depends on the diameter of the targeted portion of the nerve (see FIGS. 10 and 11). Importantly, this is time of exposure in that temperature range continuously as a single freeze (vs. protocols that alternate freeze and thaw cycles). External factors that change the temperature, as above, will change the amount of time the probe will need to function, not the amount of time the nerve experiences the appropriate temperatures. Only measurement of the temperatures in vivo will correlate with the stated times. FIGS. 10 and 11 assume that the probe is placed perfectly adjacent to the anatomical target. It should be understood that placing the probe perfectly adjacent to the target may not be realistic depending on the procedure and/or anatomy. In other words, the probe may be spaced apart from the target during the procedure. As a result, longer exposure times may be needed, which may lead to more non-target effects and unwanted damage (see FIG. 12). This disclosure contemplates that the systems, devices, and methods described herein, which allow for real-time measurement and feedback of local temperature and/or allow for real-time control of the cryozone, can minimize or eliminate such issues. Additionally, the risk of nontarget ablation and unwanted damage to nontarget tissues goes up rapidly with increasing time of ablation—and therefore increasing diameter of the nerve, further illuminating the value of directional gradients with real time feedback (see FIG. 12). Drawing on this knowledge, the systems described herein may include a console attached to the probe such that the probe can provide tissue temperature measurements and the console will then calculate the time a given target has been exposed to the appropriate temperature and determine a “complete ablation” time for the operator. The user interface can indicate for the operator when the ablation is complete. Further, based on temperature measurements, the system can be manually controllable such that unwanted cold temperatures threatening non-target ablation can be controlled, modified, and directed in space during the ablation.

In some implementations, this disclosure contemplates a single cryoablation treatment will be effective. In other implementations, this disclosure contemplates repeating cryoablation treatment following nerve regeneration. In most cases, if the nerve itself is damaged, the regenerated nerve may not manifest the same characteristics. For example, pudendal nerves that have been damaged during gynecological interventions or as a result of chronic bike-riding or horseback riding undergo mechanical stretching and/or compression. Neuromas that form following amputation create a plasticity and “windup” related to peripheral nerve scar tissue traction, compression of residual nerves, ischemia, and/or peripheral upregulation of ectopic ion channels contributes to unpleasant sensations that localize to the deafferented body part. The microenvironment about a peripheral axotomy induces biochemical changes that result in increased expression of voltage-sensitive sodium channels, decreased potassium channel expression, altered transduction molecules involved in mechano-, heat, and cold sensitivity, increased concentrations of inflammatory mediators, and altered adrenoreceptor subtype expression—the end product of which are ectopic action potentials. These “firings” have been characterized and implicated in the establishment of ongoing noxious signals, intensification and summation effects on ectopic signals from the DRG, central nervous reorganization, and global neuraxis sensitization, not to mention the pain itself. In both of these cases, and other similar clinical scenarios, the nerve undergoes Wallerian degeneration and subsequent axonal regeneration—the end product of which is essentially a “new nerve.”

On the other hand, when nerves are cryoablated appropriately in the setting of existing extrinsic disease, such as in the cases of knee osteoarthritis or diabetic peripheral neuropathy, the regenerated nerve will resume signaling related to the unchanged condition—in these examples advanced osteoarthritis or peripheral vascular disease. In these cases, the procedure may be repeated, as it is clear from preclinical research that repeat ablations do not negatively affect regeneration potential. In contradistinction, though, pain related to peripheral neuropathy caused by a single insult (chemotherapy or noxious stimuli, for example) or pain related to osteoarthritis of the knee that is subsequently replaced, will result in regenerated “new nerves” that do not transmit painful stimuli.

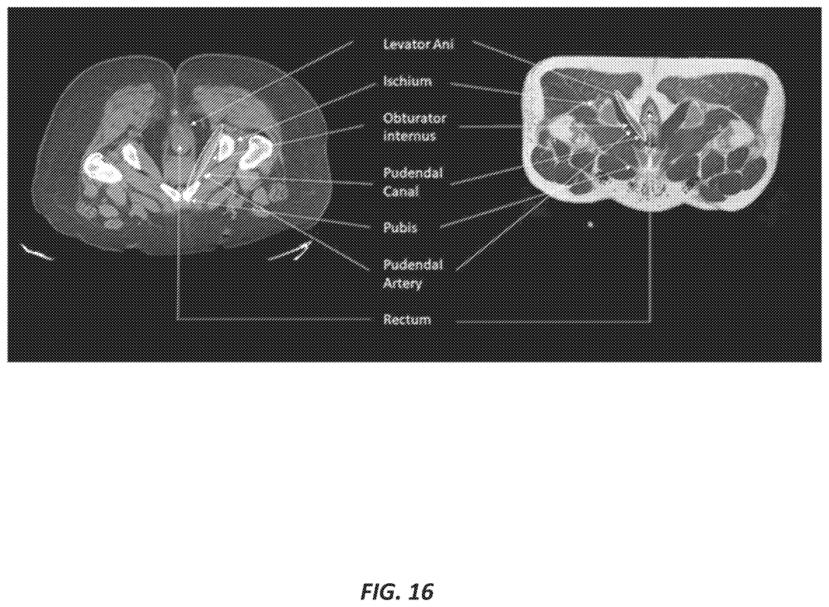

The target tissue depends on the disease state. In every case, though, advanced imaging guidance techniques (CT, MRI, ultrasound) are required to safely access the target, and control of the ablation zone with real time informational feedback is necessary to avoid non-target ablation and to obtain uniform, precise inclusion of the nerve. Implementing interventional radiology skills and advanced imaging guidance allows for a myriad of novel nerve targets. The targets are deep structures in the body, surrounded by vital organs and vessels, that are not accessible non-surgically without advanced imaging guidance and interventional radiology training. (FIG. 16).

Placement of the probes are specific for each disease state, as are the specific times of cold temperature exposure. In the case of obesity and poor diet adherence, the target is the posterior vagal trunk as it transitions to a plexus at the distal esophagus and gastroesophageal junction. In fact, interruption of subdiaphragmatic vagus nerve signaling has long been associated with loss of appetite in humans, as well as weight loss or attenuation of weight gain in all species studied. Surgeries that interrupt or modulate vagal nerve signaling aim to diminish hunger and accelerate satiation based on afferent nerve fibers that carry signals from the gut to the brain (80-90% of vagal fibers at the gastroesophageal junction) and efferent contributions that regulate pyloric relaxation and gastric motility, respectively—but have been limited by unfavorable cost-risk-benefit ratios. An image guided, percutaneous approach allows the vagus signaling to be predictably, temporarily (8-12 months) attenuated with a single simple needle outpatient procedure. Image guidance may be necessary to safely guide the probe to the appropriate location in some procedures.

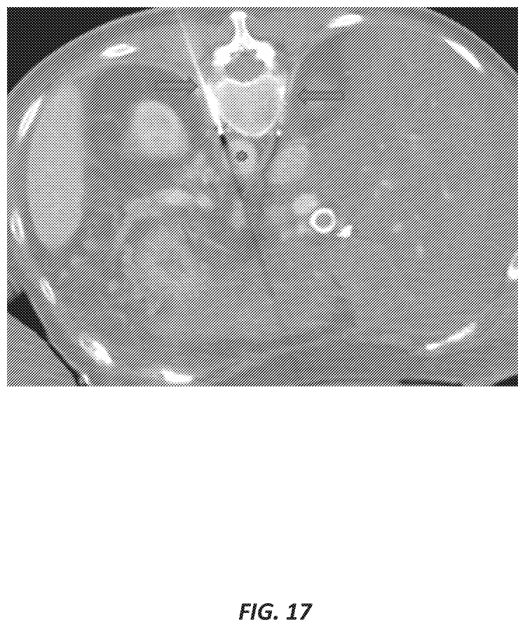

For splanchnic nerves, hyperactivity of which have been long associated with hypertension, metabolic syndrome, and obesity—CT guidance allows safe placement of cryoablation probes laterally as they course about the vertebral body. Specific image guided placement of probes that have controllable ablation zones is required to safely address the nerves and accurately ablate them. Real time temperature measurements are critical because of adjacent vasculature that changes the induced temperatures via “cold-sink.” (FIG. 17).

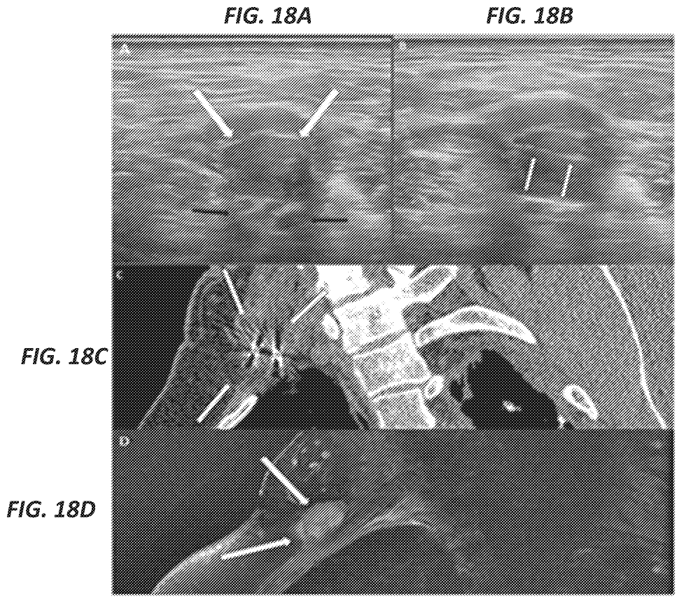

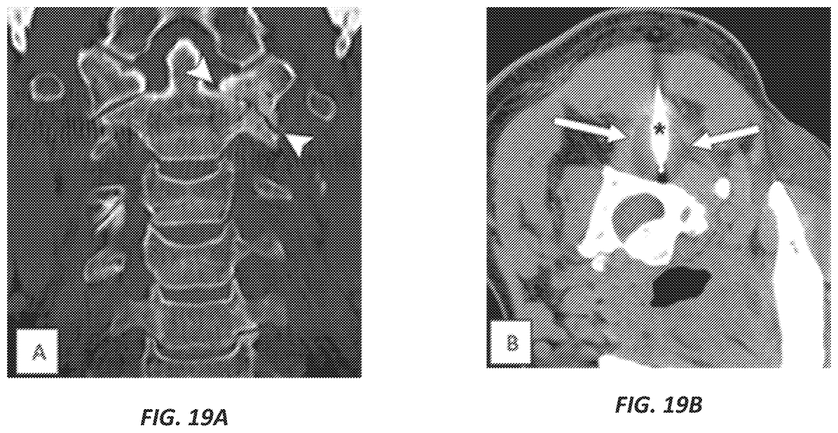

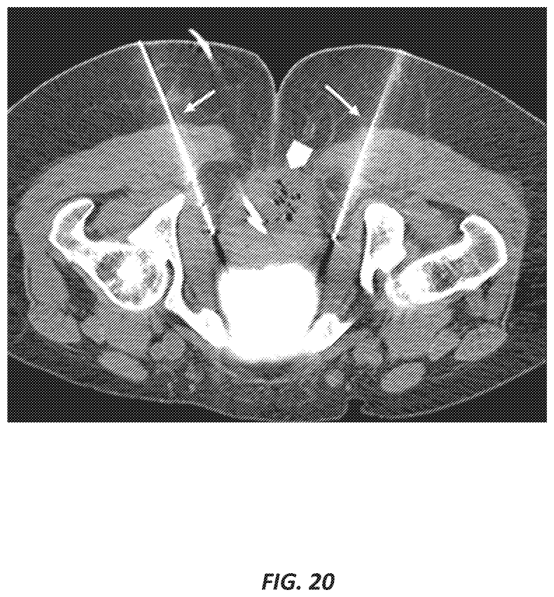

In the case of peripheral applications, the target is the pain generator. (FIGS. 18-20) As illustrative examples, in the setting of phantom limb pain the target is the neuroma or distal amputated nerve. For occipital neuralgia, the target is the greater occipital nerve as it traverses the C1-C2 plane, and for pudendal nerves the ischiorectal fat.

Percutaneous CT-guided Vagus Nerve Cryoablation (Cryovagotomy)

A percutaneous CT-guided cryovagotomy trial is described in Prologo, J. David, et al. “Percutaneous CT-Guided Cryovagotomy in Patients with Class I or Class II Obesity: A Pilot Trial.” Obesity 27.8 (2019): 1255-1265. The key is to selectively decrease the temperature of the posterior (or anterior) esophageal plexus to exactly −20 C using real time measurement of a change induced by a directional ablation zone—without damaging the esophagus. This can be done by creating an ablation zone that projects forward from the probe in a shape that conforms to the esophagus so that there are not any non-target ablation, such as below, and according to the time-temperature calculations.

Nearly three-fourths of Americans are obese or overweight. This is despite extensive evidence supporting the efficacy of negative energy balance diet programs, and more than one hundred million attempts to lose weight per 12-month period in this population.

This disparity is in part explained by low rates of adherence to available programs which would otherwise result in desired weight loss. In fact, there is little doubt that adherence is more important to obesity management than the type of diet prescribed.

The vagus nerve is one potential target for intervention to attenuate hunger and improve adherence in patients undergoing calorie restriction for weight loss. In fact, interruption of subdiaphragmatic vagus nerve signaling has long been associated with loss of appetite in humans, as well as weight loss or attenuation of weight gain in all species studied. Surgeries that interrupt or modulate vagal nerve signaling aim to diminish hunger and accelerate satiation based on afferent nerve fibers that carry signals from the gut to the brain (80-90% of vagal fibers at the gastroesophageal junction) and efferent contributions that regulate pyloric relaxation and gastric motility, respectively—but have been limited by unfavorable cost-risk-benefit ratios.

At the same time, the evolution of advanced imaging guidance and cryoablative technology has led to new percutaneous options for a variety of historically difficult to treat clinical conditions related to nerves. Specifically, cryoneurolysis (application of cold to nerves using small gauge, closed-end needle systems) results in a well characterized, local, reversible nerve signaling attenuation that can be delivered as a single puncture outpatient procedure.

Presented below are the results of a pilot study designed to, 1) evaluate the safety and feasibility of CT-guided percutaneous cryoablation of the vagus nerve (percutaneous cryovagotomy) in the setting of obesity, and 2) derive estimates of key study parameters to support randomized controlled trial design. Secondary outcomes reported include weight loss, quality of life, dietary intake, global impressions of hunger change, activity, and body composition analysis following the procedure.

The study was an open-label, single-group (non-randomized) pilot investigation. Stopping criteria for the trial were established a priori with the intention of minimizing the number of patients undergoing a procedure with an unknown safety profile and ensuring awareness of unacceptable rates of adverse events with as few patients as possible. The stopping criteria of the trial were: (1) 3 of the first 8 patients experiencing a Grade 3 procedure-related adverse event (AE) or procedure-related severe adverse event (SAE) at any point during the 24-hour post-procedure follow-up, (2) 4 participants experiencing a Grade 3 AE at any time post-procedure, and (3) a Grade 4 AE, Grade 5 AE, or SAE being experienced by a patient at any point during the trial. Since the data collected from the trial was intended to be used to inform the design of a subsequent study investigating efficacy if percutaneous cryoablation for weight loss was demonstrated to be feasible and safe in the current study, it was determined that the trial would only terminate early following violation of these safety criteria.

Subjects were recruited from five sites within a large health system that serves racially, ethnically, and economically diverse populations.

Each patient underwent a total of 6 in-office visits, consisting of the initial screening visit, the baseline/procedure visit, and 4 follow-up visits at 1 week (7 days), 6 weeks (45 days), 3 months (90 days), and 6 months (180 days) post-procedure. Feasibility and procedure-related safety outcomes were assessed at the baseline/procedure visit and outcomes related to post-procedure safety were collected for each patient throughout the trial. Weight loss endpoints were measured at baseline and all follow-up visits, while endpoints related to physical activity, health-related quality of life, and dietary intake were measured at baseline and the terminal visit at 6 months post-procedure.

All ablations were performed under conscious sedation induced with intravenous midazolam (Hospira) and fentanyl (West-Ward Pharmaceuticals, New Jersey, USA). The patients' vital signs were continuously monitored by a radiology nurse. With the patient prone on the CT scanner (GE Lightspeed VCT 64, New York), serial axial unenhanced images were acquired of the thoracolumbar region to include the abdominopelvic junction, and the region of the posterior vagal trunk and/or plexus was identified.

Following tract anesthesia with 1% Lidocaine (Hospira, North Carolina, USA), a 1.7 mm diameter cryoablation probe (Endocare, Texas, USA) was advanced to the region of the posterior vagal trunk as it transitions to a plexus along the posterolateral esophagus on the right. With the probe in position, two 2-minute freeze cycles were undertaken, separated by a 1-minute passive thaw—according to established cold induced nerve injury models. The probe was then removed following a second passive thaw period of 1 minute. The patients were recovered for 60-90 minutes after the procedure per institutional moderate sedation protocol, then discharged.

Feasibility was measured by the technical success rate of the cryoablation procedures. Technical success was defined as successful placement of the cryoablation probe percutaneously, using CT guidance, such that the posterior vagal trunk was included in the predicted ablation zone. In addition, concluding technical success for a procedure required that no procedure-related AEs had occurred.

Safety was quantified by the rate of procedure-related events (AEs occurring within 24 hours following the procedure), breakthrough events (AEs occurring at any time that required emergency or urgent physician consultation), AEs, and/or SAEs. Specific clinical signs or symptoms that defined AEs for these criteria were (amongst other potential Grade 3-5 AEs not listed here), constitutional symptoms (severe fatigue interfering with ADLs, fever >40° C., prolonged and/or severe rigors), endocrine (insulin requiring glucose intolerance, ketoacidosis), gastrointestinal (inadequate caloric intake requiring TPN or IV fluids, diarrhea requiring IV fluids and/or manifesting as >7 stools/day, symptomatic abdominal distention or bloating, severe abdominal pain requiring narcotics, ileus, severe nausea requiring hospitalization, bowel obstruction or perforation), hemorrhage requiring intervention, infection requiring antibiotics, or pain interfering with activities of daily living.

Total body weight was recorded prior to the procedure and at each follow-up visit. Calculation and reporting of metrics related to weight loss followed recommendations established by the American Society for Metabolic and Bariatric Surgery for standardized outcomes reporting in metabolic and bariatric surgery. Weight loss metrics included: (1) absolute weight; (2) BMI, [kg/m²]; (3) percent total weight loss, “TWL” [((Initial Weight)−(Postop Weight))/[(Initial Weight)]; (4) percent excess weight loss, “EWL” [((Initial Weight)−(Postop Weight))/((Initial Weight)−(Ideal Weight))]; and (5) Percent excess BMI loss, “EBMIL” [((Initial BMI)−(Post-procedure BMI))/(Initial BMI−25)]. All instances of ideal weight were derived from Metropolitan Life tables, in which ideal weight is defined by the weight corresponding to a BMI of 25 kg/m′.

Quality of life was measured using the Moorehead-Ardelt quality of life questionnaire II (MA-II). The MA-II is a six-item questionnaire on which subjects rank their quality of life as it relates to general self-esteem, physical activity, social contacts, work satisfaction, sexual pleasure, and focus on eating behavior—and is part of the Bariatric Reporting and Analysis Reporting Outcome System.

Dietary intake data was quantified prior to the procedure and at terminal follow up using the Nutrition Assessment Shared Resource of the Fred Hutchinson Cancer Research Center food frequency questionnaire (FFQ). Subjects indicate frequency and portion size of meals and snacks over time, and software analysis translates responses to overall caloric intake, as well as macronutrient distribution breakdown.

Changes in patients' perception of hunger before and after cryoablation was quantified using a Patient Global Impression of Change (PGIC) scale. The PGIC is a comprehensive, single-item subject estimate tool validated across specialties to assess treatment related improvement and patient satisfaction following intervention. Patients were asked to rate their change in appetite post-procedure using a 7-point scale that ranged from: very much less, much less, somewhat less, no change, somewhat more, much more, and very much more.

Physical activity was measured using the Kaiser Physical Activity Survey (KPAS). The KPAS instrument is specifically designed to include activity related to housework/caregiving, sports/exercise, active living habits, and occupation activities. The KPAS was administered as a paper questionnaire prior to the procedure and at terminal follow up. Subjects indicated their level of participation in activities ranging from “never” to “always,” wrote in their occupation and ranked activity variables related to occupation and wrote in answers to questions that queried for involvement with leisure sports and activity exercise. The questionnaire is scored according to subject answers, and incorporation of specific activity index variables to account for variable effort across activity domains.

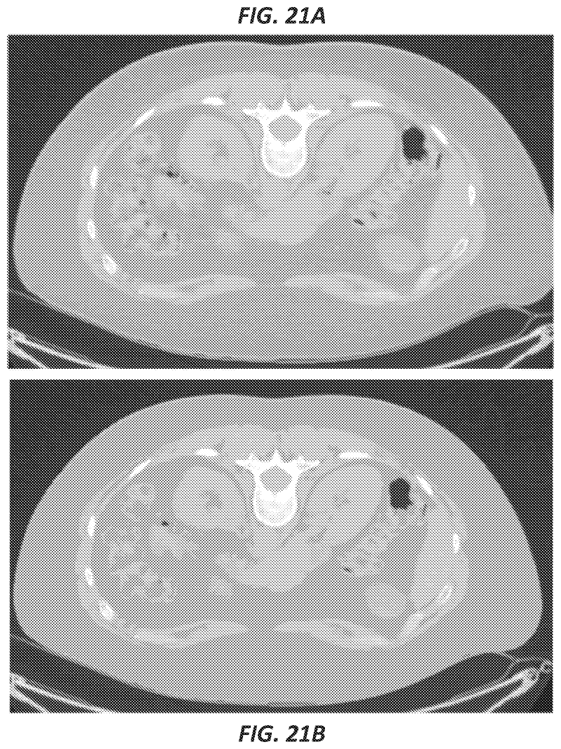

Body composition was measured using CT during the procedure and at terminal follow up, according to established methods. Specifically, from each procedure image set, an axial slice that crossed the L1 center was identified. The ribs were followed in a slice roam viewing function to determine the slice location of T-12, and L1 centered in a 3-plane reformat view. Bi-modal regional histograms of the unfiltered pixel data were analyzed visually to obtain image intensities of bordering tissues. Intensity thresholds were centered between histogram peaks to reduce partial volume errors and applied globally across the slice. Intensity thresholds were determined for boundaries of air/skin, fat/organ tissue, and air/organ tissue. Interactively seeded threshold masks were obtained for evaluating total body cross-section area and total fat area. Using morphological image operations on the binary fat image, with incidental manual paint/erase correction, a mask of subcutaneous fat was generated. Visceral fat area was calculated by subtraction. (FIGS. 21A-21B)

Using the package “OneArnnPhaseTwoStudy” for R (R Core Team.Vienna, Austria: R Foundation for Statistical Computing), a flexible two-stage, single-arm trial was planned around the stopping criteria based on Simon's optimal two-stage approach with the design modifications described by Kunz & Kieser. Using this design, the total number of patients needed to conclude the safety and feasibility of percutaneous cryoablation with an a of 0.05 and a power of at least 80% was 20, with 8 patients required for Stage 1 and an additional 12 patients expected to be enrolled in Stage 2.

Analyses on metrics related to weight loss, post-procedure perception of hunger, physical activity, quality of life, and dietary intake employed a linear mixed-effects modelling approach to perform repeated measures analyses, specifically for its ability to accommodate various characteristics of the data, such as timepoints spaced at uneven intervals, unique responses to treatment for individual patients, and correlations in measurements across time.

Based on previous studies evaluating weight loss interventions, mixed-effects models applied to repeated measures of absolute weight, BMI, and derived metrics over time included parameters for sex (female, male; self-reported), time (duration since baseline), baseline height, and baseline BMI; models for changes in BMI, used a parameter for baseline weight instead of baseline BMI. Collinearity diagnostics were performed on the predictors included in the initial model by examining variance inflation factors, coefficients of multiple correlation (R²), and condition indices using the collin command with Stata 14 software (StataCorp. 2015. Stata Statistical Software: Release 14. College Station, Tex.: StataCorp LP). For multipart instruments (KPA and MA-II), the same model was used except analyses began with evaluation of changes in the overall score and if a statistically significant change was found, each of the instrument's domains or questions was analyzed individually.

A linear exponent autoregressive correlation (LEAR) variance-covariance structure, which parsimoniously accommodates unequally spaced measurement intervals, was used for modeling weight loss metrics that were collected at all follow-up visits. Measurements collected at only the baseline and final visit were fit with a first-order autoregressive variance-covariance structure. Models were fit using restricted maximum likelihood estimation and Kenward-Roger degrees of freedom using SAS/STAT software, Version 9.4 maintenance release 5 (SAS/STAT 14.3) of the SAS System for Windows (Copyright 2018, SAS Institute Inc., Cary, N.C., USA). Residual diagnostics were performed on models by examining residuals vs. predicted means plots, residual vs. normal distribution quantile-quantile plots, and probability distribution of Pearson-type and (internally) studentized residuals.

Primary and secondary analyses were performed using the intent-to-treat population. Sensitivity analyses consisted of repeating the primary and secondary analyses including patients that had completed all assessments at all timepoints. To evaluate the impact of missing data, multiple imputation was performed using a data augmentation algorithm for continuous variables under the multivariate normal model. Responder analyses were performed post hoc on outcome measures found to be statistically significant using an anchor-based approach to define a “responder” to the treatment. The anchor, which linked changes in outcome measures to a validated instrument capable of measuring meaningful qualitative changes, were PGIC answers of “much less” or “very much less” at the terminal visit (referred to as a “reduction in appetite” herein).

Values estimated from mixed-effect models or calculated for hypothesis testing are reported as “mean (Lower 95% C.I. Bound, Upper 95% C.I. Bound; p-value)”. All statistical analyses were performed using an a of 0.05. All figures were produced using OriginPro 2019 (OriginLab, Northampton, Mass., USA).

Of the 100 patients screened, 22 patients provided informed consent, of which 20 patients underwent the cryoablation procedure. Of these 20 patients, only 18 completed all assessments, due to one patient being lost to follow-up after the 3-month post-procedure follow-up visit and another patient failing to return for any of the post-procedure follow-up visits except the final visit at 6-months post-procedure. All subjects had a documented body mass index (BMI) 30 and 37, were years of age, and reported previous failed weight loss attempts.

Percutaneous cryoablation was performed without procedure-related complications in all 20 patients, corresponding to a technical success rate of 100% (86.1%, 100%). Similarly, at 6 months post-procedure, there were no reports of breakthrough events, AEs, or SAEs from any of the 19 patients that completed the trial, corresponding to an adverse event-free response rate of 95% (78.4%, 98.2%).

Data from the current study were acquired for purposes of deriving key parameters to inform the design of a randomized, parallel-armed, sham-controlled trial evaluating efficacy. This follow-up study would have the primary objective of evaluating differences in weight loss after 1 year between patients who undergo percutaneous vagotomy to subjects undergoing a sham procedure.

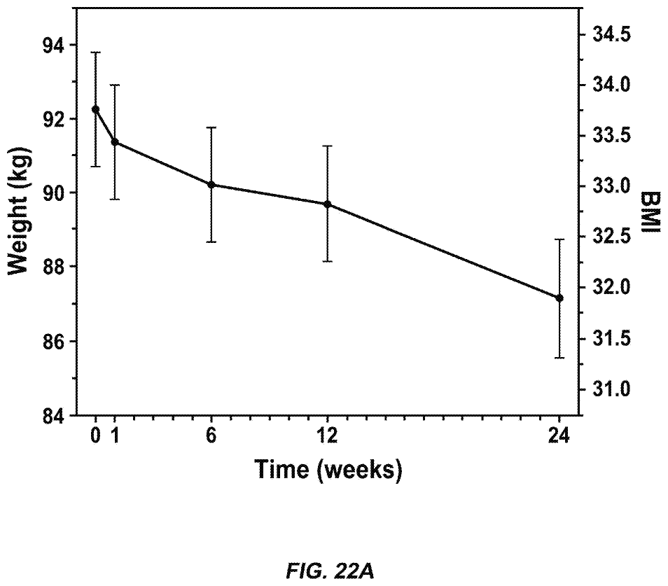

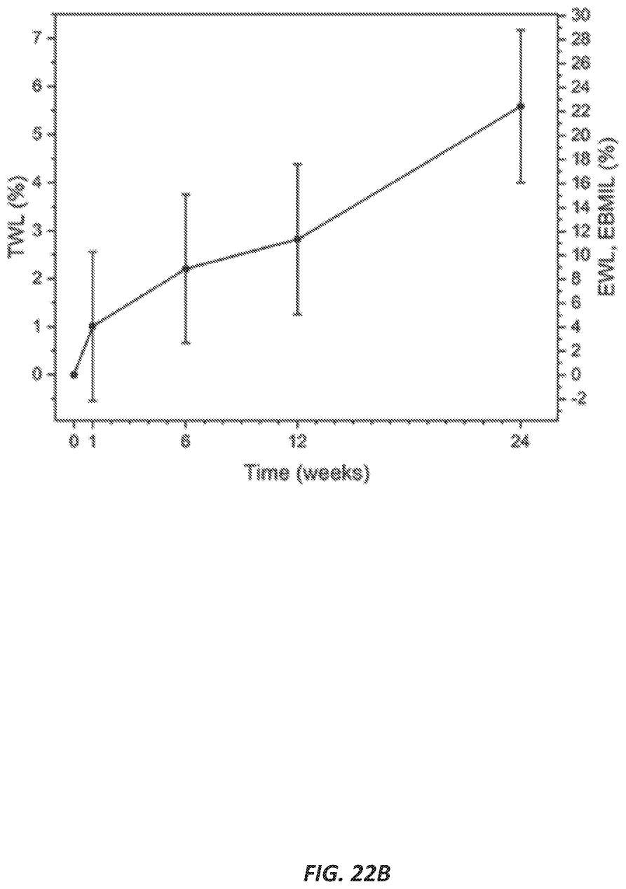

Compared to baseline values, there were statistically significant mean reductions in absolute weight observed at all timepoints. (FIGS. 22A-22B). The mean reductions in absolute weight at 1 week, 6 weeks, and 3 months post-procedure were 0.89 kg (0.22 kg, 1.6 kg; p=0.0114), 2.0 kg (0.9 kg, 3.2 kg; p=0.0007), and 2.6 kg (1.1 kg, 4.0 kg; p=0.001), respectively. By 6 months post-procedure, the mean decrease in absolute weight was 5.1 kg (3.3 kg, 6.9 kg; p<0.0001), with 45.3% of patients experiencing at least a 5 kg decrease and 13.4% of patients experiencing at least a 10 kg decrease compared to their baseline weight; 50% of the patients experienced at least a decrease in absolute weight compared to baseline of 3.9 kg. The proportion of responders who reported a post-procedure reduction in appetite and experienced weight loss compared to baseline was 66.7% and their mean reduction in absolute weight was 5.2 kg (4.1, 5.8), corresponding to a mean difference in absolute weight loss between the whole group and the responders of −0.1 kg (−2.2, 2.0) that was not statistically significant.

The mean reductions in BMI at 1 week, 6 weeks, and 3 months post-procedure were 0.33 (0.08, 0.58; p=0.011), 0.75 (0.33, 1.2; p=0.0007), and 0.94 (0.41, 1.5; p=0.0008) points, respectively. By 6 months post-procedure, the mean decrease in BMI was 1.9 (1.2, 2.5; p<0.0001), with 50% of patients experiencing at least a 1.7-point decrease, 43% of patients experiencing at least a 2-point decrease, 16.6% of patients experiencing at least a 3-point decrease in BMI compared to baseline. The results from sensitivity analyses on only patients with complete data sets and with imputed missing data produced equivalent results to those from the primary analysis and are thus not reported.

Correspondingly, the statistically significant changes in weight were reflected in the derived metrics percentage of total weight loss (TWL), percentage of excess weight loss (EWL), and percentage of excess BMI loss (EBMIL) as early as 6 weeks post-procedure. At 6 weeks and 3 months post-procedure, mean TWL was 2.2% (0.6%, 3.8%; p=0.0091) and 2.8% (1.2%, 4.4%; p=0.0014), respectively, while mean EWL and EBMIL were 8.8% (2.7%, 14.9%; p=0.0066) and 11.5% (5.3%, 17.8%; p=0.0007), respectively. By 6 months post-procedure, the mean TWL was 5.6% (3.9%, 7.2%; p<0.0001), with 50% of patients experiencing TWL of at least 5.2%, 50.8% experiencing TWL of at least 5%, and 15.7% of patients experiencing TWL of at least 10%. Similarly, the mean EWL and EBMIL at 6 months post-procedure were 22.7% (16.4%, 29.1%; p<0.0001), with 50% of patients experiencing EWL/EBMIL of at least 18.6%, 46.6% experiencing EWL/EBMIL of at least 20%, and 32.7% of patients experiencing EWL/EBMIL of at least 30%.

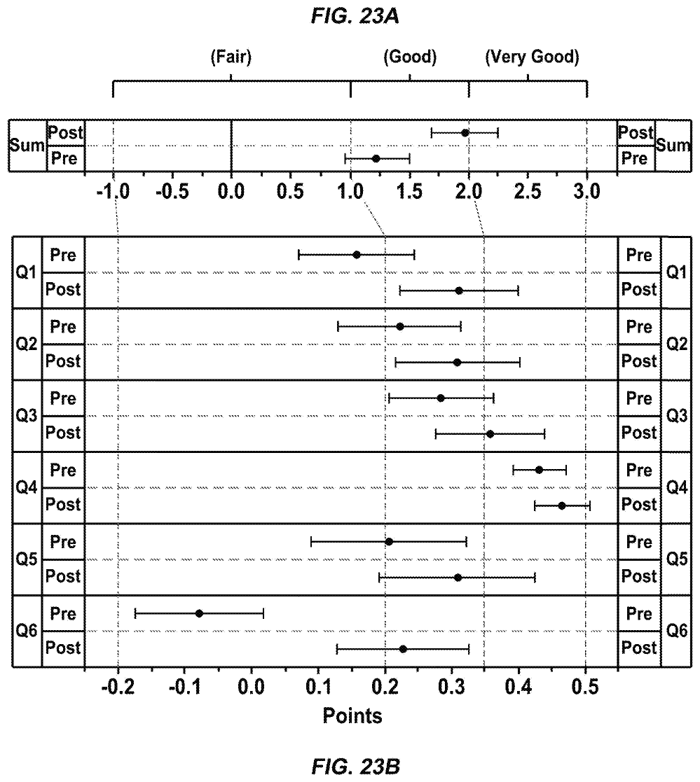

There was a statistically significant increases in mean MA-II score of 0.75 points (0.41, 1.1; p=0.0002) from baseline to 6 months post-procedure. Considering only responders, 86.7% of patients who reported reductions in appetite post-procedure appetite had a mean increase in MA-II quality of life score at 6 months post-procedure of 0.62 points (0.24, 0.99). Comparing the mean increase in quality of life of the whole group to that of the responders, there was a mean difference of 0.13 points (−0.2, 0.5) that was not statistically significant. (FIG. 23A)

Investigating each of the six questions that comprise the MA-II questionnaire individually, at 6 months post-procedure there were mean score increases compared to baseline, however not all were statistically significant. At 6 months post-procedure there were statistically significant score increases for the questions “Usually I Feel . . . ”, “I Enjoy Physical Activities . . . ”, and “The Pleasure I get Out of Sex Is . . . ”, of 0.15 points (0.05, 0.25; p=0.0042), 0.09 points (0.02, 0.16; p=0.0161), and 0.10 points (0.01, 0.20; p=0.033), respectively. The question that had the greatest change in score was “The Way I Approach Food Is . . . ”, which had a mean of −0.1 points (−0.18, 0.02) pre-procedure and increased to 0.23 points (0.13, 0.33) points post-procedure, representing a statistically significant increase of 0.30 points (0.19, 0.42; p<0.0001) and a qualitative shift from “fair” to “good” on the MA-II's quality of life scale. As evidence of internal consistency between the two quality of life measures, the patient who reported that their appetite had not changed since pre-procedure via the PGIC had no change in their score for question 6 on the MA-II.

Based on information gleaned from FFQs, daily estimates of dietary caloric intake were computed and rounded to the nearest 10-unit for reporting purposes. Pre-procedure, dietary caloric intake was 1900 Calories (1560, 2250) and had decreased to 1290 Calories (950, 1640) post-procedure, representing a statistically significant mean decrease of 610 Calories (210, 1010; p=0.005). (FIG. 23B) At 6 months post-procedure, 50% of patients had a daily caloric intake deficit of at least 460 Calories, 47.7% had a deficit of at least 500 Calories, and 23.9% had a deficit of at least 1000 Calories. Considering only responders, 78.6% of the patients who reported post-procedure appetite as being “very much less” or “much less” on the PGIC experienced post-procedure had mean daily caloric intake deficit of 640 Calories (160, 1130), corresponding to a mean difference in daily caloric intake compared to the whole group of −30 Calories (−490, 430) that was not statistically significant.

At 6 months post-procedure, 95% of patients reported using the PGIC that they felt that their appetite was less than it was pre-procedure, while one patient reported that their appetite was unchanged. It was noted that by 6 months post-procedure, the patient that reported no change in their appetite had an estimated daily caloric intake deficit of 190 Calories and had experienced an absolute weight loss of 3.9 kg, which coincidentally corresponded to a TWL of 3.9%. Of the rest of the patients that reported a decrease in their appetite post-procedure, 15.8% reported that their appetite was “somewhat less”, 68.4% reported that their appetite was “much less”, and 10.5% reported that their appetite was “very much less” compared to pre-procedure.

At 6 months post-procedure, 84% of patients reported increases in physical activity levels as measured using the KPAS. From baseline to 6 months post-procedure, there was a statistically significant increase in mean KPA score of 2.3 points (1.0, 3.6; p=0.0009), corresponding to a 22% increase in pre-procedure activity levels. Considering only responders, 86.7% of patients who reported reductions post-procedure appetite had a mean increase in physical activity levels at 6 months post-procedure of 1.7 points (0.7, 2.6), which was a mean of 0.6 points (−0.8, 2.0) less than the mean increase in physical activity levels experienced by the whole group but was not statistically significant.

Individual evaluation of the four domains comprising the KPAS revealed statistically significant score increases in all four domains. At 6 months post-procedure, there was a statistically significant increase in “Household and Family Care” activities of 0.27 point (0.02, 0.52; p=0.0384) and in “Occupational” activities of 0.28 point (0.11, 0.45; p=0.0036). The activity domains “Active Living Habits” and “Participation in Sports and Exercise” had the greatest increases from pre- to post-procedure of 0.61 points (0.18, 1.0; p=0.0086) and 1.1 points (0.52, 1.7; p<0.0011), respectively; these increases represented a 24.4% increase in “Active Living Habits” and a 43.1% increase in “Participation In Sports and Exercise” at 6 months post-procedure compared to baseline levels.

At 6 months post-procedure, 73.7% of patients experienced a reduction in body fat percentage with a statistically significant mean reduction in body fat of 4.1% [0.47, 6.0; p=0.0245). Compared to their body fat percentage at baseline, 68.4% of patients experienced a decrease in body fat percentage of at least 2.5% and 31.6% experienced a decrease of at least 5% at 6 months post-procedure. Considering only responders, 80% of patients who reported a reduction in appetite post-procedure experienced a mean decrease in body fat percentage at 6 months post-procedure compared to baseline of 3.3% (0.01, 6.6). The difference in mean reduction in body fat at 6 months post-procedure between the whole group and responders was −0.1% (−3.2, 3.0) and not statistically significant.

In this cohort, there were no procedure related complications or adverse events during a six-month trial investigating percutaneous CT guided cryovagotomy in patients with Class I or Class II obesity. Technical success was 100%, defined as the ability to place a cryoablation probe in proximity of the target nerve with the intention of performing cryoneurolysis according to established protocols. Ninety-five percent of patients reported decreased appetite following the procedure, and reductions in mean absolute weight and BMI were observed at all timepoints. The mean quality of life and activity scores improved from baseline to 6 months post-procedure, and mean caloric intake decreased over the same period.

The impetus behind this study is a potential role for CT-guided percutaneous cryovagotomy as a non-surgical adherence aid for patients following calorie restriction weight loss programs via decreased hunger. Several other studies have also investigated interventions to modify hunger, appetite, and/or the drive to eat during energy restriction which consistently demonstrate an inverse relationship between degree of hunger and weight loss success. For example, Nickols-Richardson, et. al. reported a significant decrease in self-reported hunger to 6 weeks for subjects randomized to a high protein/low carbohydrate diet, compared to a high carbohydrate/low fat diet arm, using a hunger subscale from the three-factor eating questionnaire. Subjects randomized to the high protein arm reported a 6.3±4.1 decrease in perceived hunger from baseline to week 6, compared to 3.2±2.4 in the high carbohydrate arm. Vogels, et. al. evaluated the subjective feeling of hunger using the same instrument during maintenance phase following a very-low-calorie diet. Subjects who were successful in maintaining their weight loss had significantly less hunger than those who were not (−4.0±4.9 vs. −1.2±2.7, respectively).

Johnstone, et. al. used a 100 mm visual analog scale (VAS) method to record subjects' perceptions of hunger intensity hourly during waking hours, and found a significant difference between those on a low carbohydrate-ketogenic arm (less hungry [16.8 mm]) vs. a medium carbohydrate non-ketogenic diet (more hungry [21.4 mm]). Drapeau, et. al. used a 150 mm VAS to measure “appetite sensations” determined by compiling responses to several questions, including “how hungry do you feel.” One hour post prandial scores in this study were predictive of subsequent energy intake in subjects who were actively trying to lose weight.

In this cohort, 95% of patient responses throughout the follow up period indicated that their appetite was less than it was perceived to be prior to the procedure. Increases in physical activity and quality of life scores, as well as decreases in caloric intake and overall body fat are internally consistent with the notion that decreased hunger may improve adherence to healthy living schedules.

With regard to surgeries that involve the vagus nerve, several investigators have evaluated surgically implantable vagal neuromodulation devices that use electrical stimulation to block neural activity. The procedure involves implanting a subcutaneous electrical device that is connected to the vagal trunks by laparoscopically placed electrodes. The device is transcutaneously controllable and rechargeable. It delivers low energy pulses at high frequencies for fixed intervals intended to intermittently block vagal signaling for purposes of increasing satiety and reducing hunger.

Subsequent large randomized trials of vagal blockade using implantable devices have consistently reported statistically significant EWL in the treatment groups, but not always significantly more than control arms. Apovian et. al. explicitly measured effect of vagal blockade on hunger in 123 subjects who underwent implantation and therapy for 24 months. They reported a significant mean decrease using the three-factor questionnaire in perceived hunger of −4.1 from screening at 12- and 24-months post procedure.

The mechanism of cryoablation induced vagal blockade differs in that exposure of nerves to cold results in cessation of nerve conduction, development of endoneural edema, and subsequent Wallerian degeneration from the point of injury, distally. The endoneurium and myelin sheath are left intact, and in combination with Schwann cells, provide scaffolding and direction for predictable axonal regeneration at a rate of 1-2 mm/day. Also, the procedure differs from surgical vagal interventions in that the delivery of therapy can be accomplished percutaneously with a needle during a one-time outpatient procedure, which may positively affect unfavorable cost-risk-benefit ratios currently limiting clinical translation of surgical vagal interruptions.

This study demonstrates the feasibility of percutaneous CT-guided cryovagotomy in patients with body mass indices from 30-37, and provides quantitative preliminary data that informs the design of a larger, parallel-armed, sham-controlled, randomized clinical trial to investigate changes in total weight loss between patients receiving cryoablation of the vagus nerve and patients undergoing a sham procedure.

Additionally, this disclosure contemplates that the cryoablation probes and/or systems described with respect to FIGS. 1-8A and 15 can be used to perform a cryovagotomy procedure. Such probes and/or systems provide advantages and/or improvements as described herein as compared to conventional devices, systems, and processes. While this example demonstrates the technical feasibility of performing a cryovagotomy procedure, it does not guarantee that the patients experience clinical benefit at least because of the inability of the clinician to know the treatment temperature and/or exposure time when the procedure is performed using a conventional probe. As described herein, the cryoablation probes and/or systems described with respect to FIGS. 1-8A and 15 address this deficiency of conventional probes and systems. Moreover, the cryoablation probes and/or systems described with respect to FIGS. 1-8A and 15 facilitate real-time spatial and temporal control of cryozone(s), which allows the user/operator to target specific anatomy for treatment while minimizing or eliminating unwanted impacts on adjacent, non-target tissue.

Percutaneous CT-Guided Splanchnic Nerve Cryoablation (Cryosplanchnicetomy)

A percutaneous CT-guided cryosplanchnicetomy study is described below. The study below confirms nerve involvement by the induced ablation zone. In the case of the splanchnics, nerves can be targeted at T 12 will a medially directed gradient according to time-temperature calculations to attenuate autonomic fibers without damaging any adjacent organs. For the management of hypertension and hyperglycemia and obesity.

More than 35% of adults in the United States manifest characteristics of the metabolic syndrome—hypertension, hyperglycemia, and obesity+/−hyperlipidemia—and the worldwide prevalence is predicted to surpass 50% by 2035. It has become increasingly clear during recent years that the development and maintenance of metabolic syndrome is related to chronically increased sympathetic input to the visceral space. Moreover, interruption of the splanchnic nerve input (either bilateral or unilateral) leads to decreases in blood pressure in all species studied.

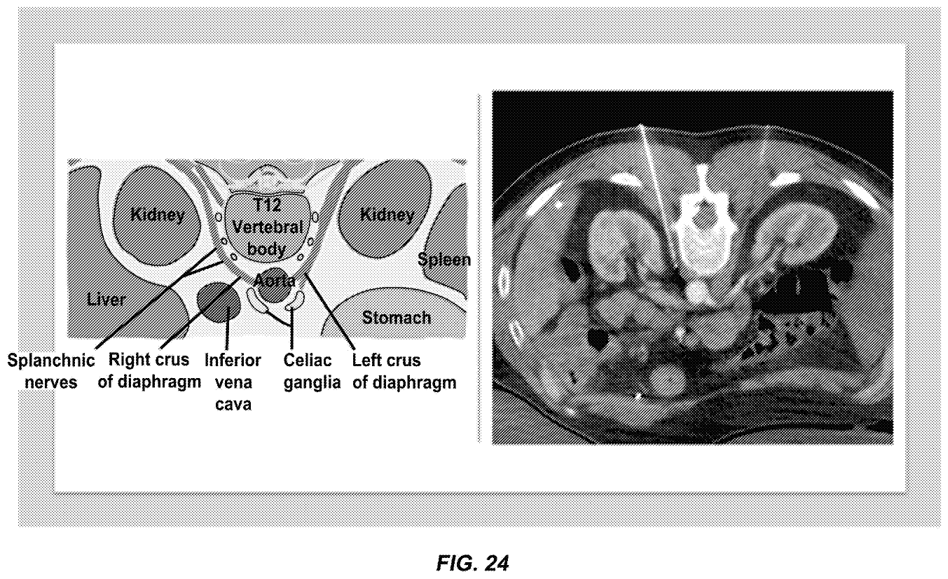

Hypertension—as part of metabolic syndrome or not—affects over 1 billion people worldwide. The role of sympathetic overstimulation in the pathogenesis and maintenance of hypertension is well documented, and includes baroreceptor and chemoreceptor set point resets, abnormal sympathetic innervation, and neurotransmitter imbalances. Based on this historical knowledge, an explosion of research has emerged in recent years around the potential ability of physicians to attenuate the sympathetic nerves involved in this process with an endovascular, catheter based approach. The results of these studies have been promising, though prospective randomized controlled trials have not proven the therapy to be clearly superior to control—almost certainly related to inability to safely and effectively interrupt nerve signaling across vessel walls in the presence of flow, without end-point feedback. Secondly, the target for these trials is peripheral and selective, which may limit global effects of the therapy. The splanchnic nerve network represents the common pathway for peripheral autonomic nerves targeted during endovascular denervation attempts and are readily interrupted using percutaneous cryoablation. (FIG. 24)

Type 2 diabetes (T2D) is a disease of pandemic proportion as well, affecting approximately 425 million adults worldwide. Unfortunately, the incidence of T2D is increasing in most countries. It is predicted that by the year 2045, 629 million adults will be diagnosed with T2D worldwide. Within the United States, 30.3 million people have T2D, accounting for 9.4% of the US population. Weight loss is the cornerstone of treatment, and has been shown to decrease risk of long term complications, lead to improvements in HbA1c and lipid levels, as well as decrease need for medications and improvements in quality of life. Unfortunately, lifestyle intervention alone is often ineffective at achieving long-term sustainable, clinically significant weight loss or improvements in A1c, and patients develop progressive loss of glycemic control over time. However, even with medication management, HbA1c levels increase by approximately 1% every 2 years. Clinical inertia, or delayed initiation of more aggressive therapies, is unfortunately a large problem and leads to further diabetes complications and increased risk of comorbidities Thus, it is clear that more sustainable, effective treatment modalities are necessary to optimize management of T2D.

There has been recent interest in the role of neural modulation of glycemic control. Specifically, research suggests that chronically elevated sympathetic activity can contribute to the development of metabolic syndrome and T2D. Recent preclinical data around splanchnic denervation leads to significant improvement in fasting glucose levels, as well as glucose tolerance as measured by oral glucose tolerance tests (OGTT). These effects are thought to be mediated by decreasing levels of catecholamines, and this likely explains the improvements in systolic blood pressure observed as well.

At the same time, nearly three-fourths of Americans are obese or overweight. This is despite extensive evidence supporting the efficacy of negative energy balance diet programs, and more than one hundred million attempts to lose weight per 12-month period in this population. The splanchnic nerves are one potential target for intervention to attenuate hunger and decrease gastric motility during calorie restriction for weight loss.

A host of groups have also addressed the concept of sympathetic denervation for management of hypertension. The idea behind these trials remains that decreasing sympathetic tone will lead to decreased systemic effects, including hypertension, hyperglycemia, and potentially obesity. Indeed, most trials appreciated a decrease of 10-15 mmHg over time in office blood pressure measurements. Recent reviews acknowledge that ambulatory measurements may be a more accurate reflection of procedure effect, and that a glaring limitation remains via inability to measure actual nerve involvement difficulties that are readily overcome with CT guided cryoablation given direct visualization of the ablation zones and proximal locations of the targets.

The application of cold to nerves results in a predictable, reproducible, reversible attenuation that can be accomplished percutaneously during a single outpatient procedure using advanced imaging guidance.

The foundation for this project is rooted in the advantage of advanced imaging guidance, which affords operators enhanced precision and improves the safety and efficacy profiles of many interventional pain procedures. In parallel, ablative technology provides, and had provided through its evolution, interventional radiologists, surgeons, and pain medicine specialists with refined tools developed primarily for the ablation of cancer. Recently, utilization of advanced imaging guidance in combination with the latest ablative technologies applied toward the treatment of new clinical syndromes has resulted in the creation of therapeutic options that can readily be applied to difficult to treat conditions. Specifically, the integration of cryoablation with CT guidance for the treatment of nerve related disorders allows for detailed evaluation of the targeted anatomy, precise placement of the treatment probe, direct visualization of the ablation zone, and minimized intraprocedural and postprocedural pain. As a result, the combination of imaging guidance and cryoablation results in minimally invasive procedures that have demonstrated improved precision, accuracy, safety, and efficacy.

Cell death following traditional cryoablation results from freezing induced through a metallic probe cooled with circulated argon. The freeze manifests first in the extracellular space—causing an osmotic gradient to form which leads to cell shrinkage. As the freeze progresses, intracellular ice crystals form and cause damage directly to organelles. Similar mechanisms result in vascular injury, inducing a coagulative cascade and eventual ischemia mediated cell damage. During the thaw phase of these procedures, water then rushes into previously shrunken cells—causing them to burst. Ablation zone tissues also incur damage through interspersed apoptosis and inflammatory injury.

Cryoablation affects nerves specifically through 1) ice-crystal mediated vasa vasorum damage and endoneural edema, 2) Wallerian degeneration, 3) direct physical injury to axons, and 4) dissolution of microtubules resulting in cessation of axonal transport. The cumulative end point of these routes of neuronal damage is a Sunderland 2 classification of nerve injury—which is followed by induced Wallerian degeneration, and a complex, reproducible, sequence of nerve regeneration at a rate of 1-2 mm/day—creating a unique situation which is valuable clinically (any untoward effect from the procedure is temporary) and from a repeatability standpoint.

This disclosure contemplates that the cryoablation probes and/or systems described with respect to FIGS. 1-8A and 15 can be used to perform a cryosplanchnicetomy procedure. Such probes and/or systems provide advantages and/or improvements as described herein as compared to conventional devices, systems, and processes. While this example demonstrates the technical feasibility of performing a cryosplanchnicetomy procedure, it does not guarantee that the patients experience clinical benefit at least because of the inability of the clinician to know the treatment temperature and/or exposure time when the procedure is performed using a conventional probe. As described herein, the cryoablation probes and/or systems described with respect to FIGS. 1-8A and 15 address this deficiency of conventional probes and systems. Moreover, the cryoablation probes and/or systems described with respect to FIGS. 1-8A and 15 facilitate real-time spatial and temporal control of cryozone(s), which allows the user/operator to target specific anatomy for treatment while minimizing or eliminating unwanted impacts on adjacent, non-target tissue.

Percutaneous Nerve Cryoablation (Pain)

A percutaneous image-guided cryoablation for the treatment of phantom limb pain study is described in Prologo, J. David, et al. “Percutaneous image-guided cryoablation for the treatment of phantom limb pain in amputees: a pilot study.” Journal of Vascular and Interventional Radiology 28.1 (2017): 24-34. Pain specific applications for a cryoablation probe designed to be placed under CT-guidance, specifically direct a cryoablation zone in space, measure tissue temperature of a target nerve, document time of uniform exposure, and calculate point of precision neurolysis. This disclosure contemplates that conditions including, but not limited to the following, can be treated with cryoablation phantom limb pain, inguinodynia, pudendal neuralgia, occipital neuralgia, visceral pain related to cancer, visceral pain not-related to cancer, peripheral neuropathy, pain related to cancer outside of the abdomen, post-traumatic pain, post-operative pain, pain related to facet hypertrophy, and knee pain. This disclosure contemplates that the cryoablation probes and/or systems described with respect to FIGS. 1-8A and 15 can be used to treat nerve pain. Such probes and/or systems provide advantages and/or improvements as described herein as compared to conventional devices, systems, and processes. While Prologo, J. D. et al. demonstrates the technical feasibility of performing cryoablation for treatment of pain, it does not guarantee that the patients experience clinical benefit at least because of the inability of the clinician to know the treatment temperature and/or exposure time when the procedure is performed using a conventional probe. As described herein, the cryoablation probes and/or systems described with respect to FIGS. 1-8A and 15 address this deficiency of conventional probes and systems. Moreover, the cryoablation probes and/or systems described with respect to FIGS. 1-8A and 15 facilitate real-time spatial and temporal control of cryozone(s), which allows the user/operator to target specific anatomy for treatment while minimizing or eliminating unwanted impacts on adjacent, non-target tissue.

Percutaneous Nerve Cryoablation (Premature Ejaculation)

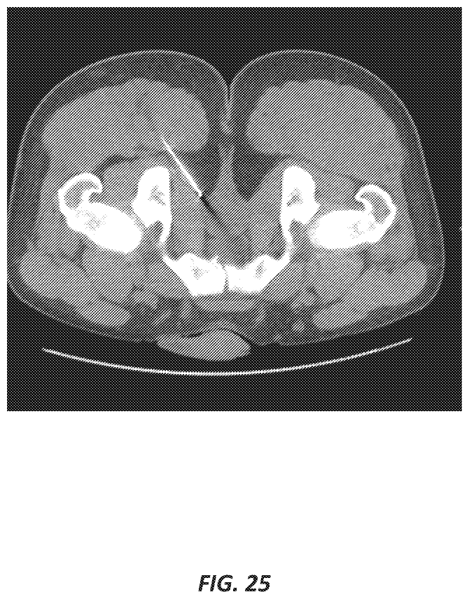

A percutaneous CT-guided cryoablation of the dorsal penile nerve for treatment of symptomatic premature ejaculation study is described in Prologo, J. David, et al. “Percutaneous CT-guided cryoablation of the dorsal penile nerve for treatment of symptomatic premature ejaculation.” Journal of Vascular and Interventional Radiology 24.2 (2013): 214-219. The CT approach to the pudendal nerve used for pain can be applied for premature ejaculation. This is a combination of two techniques. The first technique targeted the dorsal penile nerve as it emerged from the inferior pubic symphysis. Going forward, this can be combined with the data relating time of nerve exposure and temperature (see FIGS. 10 and 11) to target the pudendal nerve using CT guidance in Alcock's canal to treat premature ejaculation (see FIG. 25). This disclosure contemplates that the cryoablation probes and/or systems described with respect to FIGS. 1-8A and 15 can be used to treat premature ejaculation. Such probes and/or systems provide advantages and/or improvements as described herein as compared to conventional devices, systems, and processes. While Prologo, J. D. et al. demonstrates the technical feasibility of performing cryoablation for treatment of premature ejaculation, it does not guarantee that the patients experience clinical benefit at least because of the inability of the clinician to know the treatment temperature and/or exposure time when the procedure is performed using a conventional probe. As described herein, the cryoablation probes and/or systems described with respect to FIGS. 1-8A and 15 address this deficiency of conventional probes and systems. Moreover, the cryoablation probes and/or systems described with respect to FIGS. 1-8A and 15 facilitate real-time spatial and temporal control of cryozone(s), which allows the user/operator to target specific anatomy for treatment while minimizing or eliminating unwanted impacts on adjacent, non-target tissue.

Percutaneous Cryoablation (Cancer/Tumor)

A cryoablation for treatment of osteoid osteoma study is described in Whitmore, Morgan J., et al. “Cryoablation of osteoid osteoma in the pediatric and adolescent population.” Journal of Vascular and Interventional Radiology 27.2 (2016): 232-237. Cryoablation has gained popularity for the management of prostate cancer during the last 20 years because of, a) the often indolent nature of the disease, b) multifocality of the disease, and c) known complications of surgery. Men faced with non-life-threatening conditions often elect minimally invasive options over surgical intervention. Both urological and radiological guidelines recommend real-time monitoring during these procedures to avoid damage to the surrounding pelvic structures. As such, the current practice to insert multiple additional temperature probes at key locations. This disclosure contemplates using the devices, systems, and methods described herein to monitor temperatures with the cryoablation probe to provide a real-time map of temperature change. This obviates the need for additional punctures and temperature sensor needle placements during the procedure. As described herein, the cryoablation probes and/or systems described with respect to FIGS. 1-8A and 15 would eliminate the need to insert additional temperature sensing probes in the patient, which reduces risks of injury or infection. Moreover, the cryoablation probes and/or systems described with respect to FIGS. 1-8A and 15 facilitate real-time spatial and temporal control of cryozone(s), which allows the user/operator to target specific anatomy for treatment while minimizing or eliminating unwanted impacts on adjacent, non-target tissue.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1-22. (canceled)

23. A cryoablation probe, comprising:
a tubular member having a proximal end and a distal end, the tubular member comprising a probe tip arranged at the distal end;
a fluid channel arranged within the tubular member, wherein the fluid channel is configured to guide a thermally conductive fluid through the tubular member; and
a temperature sensor element arranged along an axial direction of the tubular member, wherein at least a portion of the temperature sensor element is configured to protrude outward from the tubular member.

24. The cryoablation probe of claim 23, wherein the temperature sensor element is configured to measure temperature in proximity to the tubular member.

25. (canceled)

26. A cryoablation system, comprising:
a cryoablation probe comprising a tubular member, a plurality of energy elements, and a plurality of sensor elements, wherein the energy elements and the sensor elements are arranged along an axial direction of the tubular member;
a fluid expansion system arranged at least partially within the tubular member, wherein the fluid expansion system is configured to circulate a thermally conductive fluid within the tubular member; and
a controller operably connected to the cryoablation probe, the controller comprising a processor and a memory, the memory having computer-executable instructions stored thereon that, when executed by the processor, cause the controller to spatially and temporally control a cryoablation zone.

27. (canceled)

28. (canceled)

29. (canceled)

30. The system of claim 26, wherein spatially and temporally controlling a cryoablation zone comprises adjusting a size, a shape, and/or a direction of the cryoablation zone.

31. (canceled)

32. (canceled)

33. The system of claim 26, wherein spatially and temporally controlling a cryoablation zone comprises steering the cryoablation zone.

34. (canceled)

35. (canceled)

36. The system of claim 30, wherein spatially and temporally controlling the cryoablation zone further comprises energizing one or more of the energy elements.

37. The system of claim 26, wherein the system further comprises a display device, and wherein the memory has further computer-executable instructions stored thereon that, when executed by the processor, cause the controller to:
receive a measurement detected by at least one of the sensor elements;
provide real-time-feedback based on the measurement detected by at least one of the sensor elements; and
display the real-time feedback on the display device.

38. (canceled)

39. (canceled)

40. (canceled)

41. (canceled)

42. (canceled)

43. The system of claim 26, wherein the cryoablation probe further comprises a sensor configured to determine position and/or orientation of the probe, and wherein the memory has further computer-executable instructions stored thereon that, when executed by the processor, cause the controller to provide information measured by the sensor configured to determine position and/or orientation of the probe to a surgical navigation system.

44-53. (canceled)

54. The system of claim 26, wherein each of the one or more energy elements is configured to convert electrical energy to heat.

55. The system of claim 26, wherein each of the one or more sensor elements is configured to measure a temperature.

56. The system of claim 26, wherein the fluid expansion system comprises a fluid channel arranged within the tubular member, wherein the fluid channel is configured to guide a thermally conductive fluid through the tubular member.

57. The system of claim 26, wherein the controller is operably connected to the fluid expansion system.

58. The system of claim 30, wherein the size, the shape, and/or the direction of the cryoablation zone is adjusted to provide the cryoablation zone in a selected angular region with respect to the cryoablation probe.

59. The system of claim 43, wherein the cryoablation probe further comprises a computer-readable identifier.

60. The cryoablation probe of claim 23, further comprising a plurality of energy elements arranged along the axial direction of the tubular member.

61. The cryoablation probe of claim 60, wherein each of the energy elements is configured to convert electrical energy to heat.

62. The cryoablation probe of claim 60, wherein the energy elements are arranged in a spaced apart relationship along the axial direction of the tubular member.

63. The cryoablation probe of claim 62, wherein a first group of the energy elements are arranged in a first circumferential region of the tubular member and a second group of the energy elements are arranged in a second circumferential region of the tubular member; or the first group of the energy elements are arranged in a first axial region of the tubular member and the second group of the energy elements are arranged in a second axial region of the tubular member.

64. The cryoablation probe of claim 60, further comprising a flexible circuit board, wherein the energy elements and the temperature sensor element are arranged on the flexible circuit board.

65. The cryoablation probe of claim 23, further comprising a plurality of temperature sensor elements arranged along the axial direction of the tubular member.

66. The cryoablation probe of claim 23, wherein the temperature sensor element is retractable.

67. The cryoablation probe of claim 66, further comprising a handle arranged at the proximal end of the tubular member, wherein the handle comprises a control mechanism configured to deploy and retract the temperature sensor element.

68. The cryoablation probe of claim 23, wherein the probe tip is a needle.

69. The cryoablation probe of claim 23, wherein the fluid channel comprises inlet and return channels for circulating the thermally conductive fluid through the tubular member.

70. The cryoablation probe of claim 23, further comprising an inertial sensor and/or a computer-readable identifier arranged along the axial direction of the tubular member.
